(19) United States
(12) Patent Application Publication
(10) Pub. No.: US 2011/0023194 A1
(43) Pub. Date: Jan. 27, 2011

(54) ENGINEERING ZYMOGEN FOR CONDITIONAL TOXICITY

(75) Inventors: Jucovic Milan, Durham, NC (US); Jeng Shong Chen, Chapel Hill, NC (US); Narenka V. Palekar, Durham, NC (US); Frederick S. Walters, Durham, NC (US)

Correspondence Address: Myers Bigel Sibley & Sajovec, PA, P.O. Box 37428, Raleigh, NC, 27627, US

(73) Assignee: SYNGENTA PARTICIPATIONS AG, Research Triangle Park, NC (US)

(21) Appl. No.: 12/747,247

(22) PCT Filed: Dec. 10, 2008
(86) PCT No.: PCT/US2008/086205

§ 371 Date: Sep. 17, 2010

Related U.S. Application Data
(60) Provisional application No. 61/012,864, filed on Dec. 11, 2007.

(51) Int. Cl. A01H 5/00; C12N 9/10; C07H 21/00; C12N 15/63; C12N 5/10; C12N 1/19; C12N 1/16

(52) U.S. Cl. 800/320.1; 435/193; 536/23.2; 435/320.1; 435/419; 435/412; 800/298; 435/254.2; 435/254.21; 435/255.1

(57) ABSTRACT

The ADP-ribosyltransferase, Vip2, exerts its intracellular toxicity in insects by modifying actin and preventing actin polymerization. Due to the nature of this toxin, expression of Vip2 in planta is lethal to the plant. Described herein are methods of making zymogens of toxic proteins that are benign in a non-target organism and are activated in a target organism. Disclosed herein are methods of engineering a random propeptide library at a terminus of a toxic protein and selecting for malfunctional variants in yeast. Using this method a selected proenzyme possesses reduced enzymatic activity as compared to the wild-type Vip2 protein, but remains a potent toxin towards corn rootworm larvae. The Vip2 zymogen can be proteolytically activated by corn rootworm digestive proteases.

### FIELD OF THE INVENTION

The present invention relates generally to the fields of biology, biochemistry and protein engineering. In particular, the present invention is directed towards zymogens of toxic proteins exhibiting conditional toxicity which are benign in a non-target host organism or cell and toxic in a target organism or cell. The present invention is further directed to methods for designing, making and using the toxins exhibiting conditional toxicity.

### BACKGROUND

Bacterial ADP-ribosylating toxins are proteins produced by pathogenic bacteria, which are usually secreted into the extracellular medium and cause disease by altering the metabolism of eukaryotic cells (Rappuoli and Pizza, 1991). ADP-ribosylating toxins break NAD into its component parts (nicotinamide and ADP-ribose) before selectively linking the ADP-ribose moiety to their protein target. In the majority of these toxins, the targets are key regulators of cellular function and interference in their activity, caused by ADP-ribosylation, leads to serious deregulation of key cellular processes and in most cases, eventual cell death.

Novel families of insecticidal binary toxins (designated Vip1 & Vip2) have been isolated from Bacillus sp. during the vegetative growth stage, where Vip1 likely targets insect gut cells and Vip2 acts as a ADP-ribosyltransferase that ribosylates actin. The Vip1-Vip2 binary toxin is an effective pesticide at 20-40 ng per g diet against corn rootworm, a significant pest of corn.

The Vip1-Vip2 complex is representative of a class of binary toxins distinct from the classical A-B toxins, such as cholera toxin, that must assemble into a complex composed of two functionally different subunits or domains for activity. Each polypeptide in the Vip1-Vip2 class of binary toxins evidently functions separately, with the membrane-binding 100 kDa Vip1 multimer presumably binding a cell surface receptor and facilitating the delivery of the 52 kDa Vip2 ADP-ribosyltransferase to enter the cytoplasm of target corn rootworm cells. Both Vip1 and Vip2 are required for maximal activity against corn rootworm. The NAD-dependent ADP-ribosyltransferase Vip2 likely modifies monomeric actin at Arg 177 to block polymerization, leading to loss of the actin cytoskeleton and eventual cell death due to the rapid subunit exchange within actin filaments in vivo. The three dimensional structure of Vip2 was solved in 1999 (Han et al., 1999, Nature Structural Biology 6:932-936). Han et al. determined that a Vip2 protein is a mixed α/β protein and is divided into two domains termed the N-domain (residues 60-265) and the C-domain (residues 266-461), which likely represent the entire class of these binary ADP-ribosylating toxins. Han et al. identified several structural features that are important in the biological activity of Vip2-like toxins including the catalytic residue at E428, the NAD binding residues at Y307, R349, E355, F397 and R400, the “STS motif” (residues 386-388) that stabilizes the NAD binding pocket, and the NAD binding pocket formed by residues E426 and E428.

As Vip2 shares significant sequence similarity with enzymatic components of other binary toxins, for example Clostridium botulinum C2 toxin (Aktories et al., 1986), Clostridium perfringens iota toxin (Vandekerckhove et al., 1987), Clostridium spiroforme toxin (Popoff and Boquet, 1987) and an ADP-ribosyltransferase produced by Clostridium difficile (Popoff et al., 1988), Vip2 represents a family of actin-ADP-ribosylating toxins.

Although the Vip1-Vip2 binary toxin has commercial potential to be a specific and potent corn rootworm control agent for use in transgenic crops, for example corn, expression of the Vip1-Vip2 complex in planta has been hampered by the fact that expression of Vip2 in cells of plants results in serious developmental pathology and phenotypic alterations to the plant itself. Therefore, there is a general need to provide methods of designing and making toxic proteins exhibiting conditional toxic activity, whereby the toxin can be rendered benign in a non-target host organism or cell as a zymogen and toxic in a target organism or cell. More specifically, there is a need to protect non-target organisms or cells expressing an ADP-ribosylating toxin, such as Vip2, from the negative effects of the toxin and yet maintain the toxic activity within a targeted living system such as an insect pest. When the non-target organism is not easily testable in a laboratory, for example a plant, there is a further need to develop a surrogate genetic system to make designing and testing zymogens more efficient.

Most naturally occurring zymogens have their propeptides localized at the N-terminus, which seems to be logical considering that synthesis of the propeptide region precedes that of the catalytic unit, thus preventing any undue activation of the zymogen (Lazure, 2002). However, it has been reported that a C-terminal pro-sequence of the subtilisin-type serine protease from Thermus aquaticus, Aqualysin I, retards the proteolytic activation of the precursor (Lee et al., 1992). However, blocking proteolytic activation does not solve the problem presented in the present invention. Here, a zymogen is needed that is benign in one living system, such as a plant but proteolytically activated in a target living system such as an insect pest that feeds on the plant.

### SUMMARY

In view of these needs, it is an object of the present invention to provide methods of designing, making, and using a zymogen of a toxic protein whereby the zymogen is benign in a non-target host organism or cell and wherein the zymogen is capable of being activated and toxic in a target organism or cell. It is also an object of the present invention to provide novel nucleic acid sequences encoding zymogens of toxic proteins which are benign in a non-target host organism or cell and which are toxic to a target organism or cell. The invention is further drawn to the novel zymogens resulting from the expression of the nucleic acid sequences, and to compositions and formulations containing the zymogens, which are benign in a non-target host organism or cell and toxic to a target organism or cell. The present invention further provides methods and genetic systems that enable efficient selection for identifying zymogen precursors wherein the toxic protein comprised in the precursor is inactive or substantially inactive.

In one aspect, the present invention provides an engineered zymogen of a toxic protein having a polypeptide chain extension fused to a C-terminus or a N-terminus of the toxic protein, wherein the zymogen is benign in a non-target organism or cell and wherein the zymogen is converted to a toxic protein when the zymogen is in a target organism or cell. In one embodiment of this aspect, the toxic protein is an ADP-ribosyltransferase. Such ADP-ribosyltransferase typically ribosylates actin of a target organism or cell.

In another aspect, the present invention provides an engineered zymogen wherein the ADP-ribosyltransferase comprises an amino acid sequence with at least 69% or 78% or 85% or 93% or 95% sequence identity to SEQ ID NO:9 and wherein the ADP-ribosyltransferase has a catalytic residue that corresponds to E428 of SEQ ID NO:9 and NAD binding residues that correspond to Y307, R349, E355, F397, and R400 of SEQ ID NO:9. In one embodiment of this aspect, the ADP-ribosyltransferase is insecticidal. In another embodiment of this aspect, the insecticidal ADP-ribosyltransferase is a Vip2 toxin. In still another embodiment of this aspect, the Vip2 toxin is selected from a group consisting of SEQ ID NO: 9, 10, 15, 16, 17, 18, and 19.

In one aspect, the present invention provides a zymogen, wherein the polypeptide chain extension comprises an amino acid sequence of at least 21 residues and having a tryptophan (Trp; W) residue at position 3, 14, and 19. In one embodiment of this aspect, the polypeptide extension comprises SEQ ID NO: 6.

In another aspect, the present invention provides a zymogen, wherein the polypeptide chain extension comprises SEQ ID NO: 8.

In yet another aspect, the polypeptide chain extension of the invention is fused to the C-terminus of the ADP-ribosyltransferase.

In another aspect, the present invention provides a zymogen, wherein the non-target organism or cell is a plant or plant cell. In one embodiment of this aspect, the plant or plant cell is selected from the group consisting of sorghum, wheat, tomato, cole crops, cotton, rice, soybean, sugar beet, sugarcane, tobacco, barley, oilseed rape, and maize.

In yet another aspect, the present invention provides a zymogen, wherein the non-target organism or cell is yeast. In one embodiment of this aspect, the yeast is Saccharomyces cerevisae.

In still another aspect, the present invention provides a zymogen comprising SEQ ID NO: 11 or SEQ ID NO: 12.

In another aspect, the present invention provides an isolated nucleic acid molecule comprising a nucleic acid sequence that encodes a zymogen of the invention; a recombinant vector comprising the nucleic acid molecule; a yeast cell comprising the recombinant vector; and a transgenic plant or plant cell comprising the recombinant vector. In one embodiment of this aspect, the yeast cell is Saccharomyces cerevisae. In yet another embodiment, the transgenic plant or plant cell is a maize plant or maize plant cell.

In yet another aspect, the present invention provides a method of making a zymogen of a toxic protein, the method comprising the steps of: a) designing a polypeptide chain which extends from a terminus of the toxic protein; b) making a library of expression plasmids which will express a zymogen precursor including the polypeptide chain upon transformation into a genetic system; c) expressing the zymogen precursor in a genetic system that is naturally susceptible to the toxic protein; d) recovering organisms or cells of a genetic system which survive step (c); e) isolating the precursor from the organisms or cells of step (d); f) testing the precursors for biological activity against a target organism or cell; and g) identifying the biologically active precursors as zymogens. In one embodiment of this aspect, the toxic protein is an insecticidal actin ribosylating ADP-ribosyltransferase. In another embodiment of this aspect, the ADP-ribosyltransferase is a Vip2 toxin. In yet another embodiment of this aspect, the Vip2 toxin is selected from a group consisting of SEQ ID NO: 9, 10, 15, 16, 17, 18, and 19. In still another embodiment of this aspect, the library comprises random amino acid sequences of at least 21 residues and having a tryptophan (Trp; W) residue at position 3, 14, and 19. In yet another embodiment of this aspect, the genetic system is a eukaryotic organism or cell. In still another embodiment of this aspect, the genetic system is yeast. In yet another embodiment, the yeast is Saccharomyces cerevisae. In another embodiment of this aspect, the target organism or cell is eukaryotic or prokaryotic. In yet another embodiment, the target organism or cell is an insect or insect cell. In still another embodiment of this aspect, the insect or insect cell is in the genus Diabrotica. In yet another embodiment, the insect or insect cell is Diabrotica virgifera (western corn rootworm), D. longicornis (northern corn rootworm), or D. virgifera zeae (Mexican corn rootworm). In still another embodiment of this aspect, the zymogen is biologically active in the target cell.

In another aspect, the present invention provides a genetic system that allows for efficient identification of an engineered zymogen precursor of a toxic protein, wherein the toxic protein in the precursor is inactive or substantially inactive and wherein the zymogen is benign in a non-target host organism or cell and is converted to a toxic protein when the zymogen is in a target organism or cell. In one embodiment of this aspect, the genetic system acts as a surrogate for a non-target organism or cell. In another embodiment, the engineered zymogen comprises a polypeptide chain extending from the C-terminus or the N-terminus of the toxic protein. In yet another embodiment of this aspect, the genetic system is yeast and the non-target organism or cell is a plant or plant cell. In still another embodiment of this aspect, the plant or plant cell is maize. In yet another embodiment of this aspect, the target organism is a pathogenic cell or organism and the toxic protein is an actin ribosylating ADP-ribosyltransferase.

In yet a further aspect, pharmaceutical compositions containing the novel zymogens of the invention are provided. Such pharmaceutical compositions should have efficacy as for example, anti-cancer agents.

Other objects, features and advantages of the invention will become apparent upon consideration of the following detailed description.

### BRIEF DESCRIPTION OF THE SEVERAL VIEWS OF THE DRAWINGS

FIG. 1 is a model of a Vip2 toxin demonstrating a propeptide concept. A: The Vip2-NAD complex illustrating NAD bound in a cleft within the C-terminal enzymatic domain of Vip2. B: Shows possible effect of an extension of a C-terminal polypeptide chain present in proVip2 (arrows 1 and 2) as interfering with the NAD binding site. Molecular graphics program WebLab ViewerPro 3.7 (Accelrys, San Diego, Calif.) was used for visualization of protein structures. Vip2 coordinates can be found in PDB database under accession number 1QS1.

FIG. 2. Is an illustration of an in vivo genetic system for selection of malfunctional Vip2 variants. Competent cells of Saccharomyces cerevisae were transformed with a plasmid carrying either a gene encoding a native Vip2 protein or an inactive Vip2 mutant (E428G). After transformation, cells were plated on plates with raffinose, providing leaky expression from a GAL1 promoter.

FIG. 3 is propeptide sequences selected after mutagenesis. Core propeptide sequence (4-4-12) selected after randomizing of 21 amino acid residues and proVip2 sequence selected after 2nd round of mutagenesis. A single nucleotide mutation (A to T) is responsible for substitution of the ninth amino acid (E to V) in the propeptide region. One nucleotide insertion acquired in a process of error-prone PCR is responsible for a frameshift and extension of polypeptide chain from 21 to 49 amino acids. Point of frameshift (*) occurred after amino acid #11 (F) of the polypeptide chain extension.

FIG. 4 is a time course of actin ADP-ribosylation with the wild type enzyme (Vip2) and its engineered proenzyme (proVip2). The ADP-ribosylation reaction was performed as described in Example 5. Aliquots were taken out from reaction at different time points and resolved by SDS-PAGE. Proteins were transferred onto PVDF membrane and ADP-ribosylated actin visualized by radiography.

FIG. 5 is a demonstration of ADP-ribosylation activity in root extract from transgenic proVip2 plant. Extraction of root proteins and ADP-ribosylation reaction were performed as described in Example 7. Aliquots of enzymatic reaction were taken out at different time points (1, 3, 5, 15, 60 minutes) and subjected to SDS-PAGE. After blotting onto PVDF membrane, ADP-ribosylated actin was visualized by autoradiography.

FIG. 6 shows the digestive fate of Vip2 proteins in western corn rootworm. Vip2 variants detected in western corn rootworm whole body homogenates after feeding for 30 and 90 minutes. Lane: 1. S-tag-proVip2 (30 min), 2. S-tag-proVip2 (90 min), 3. proVip2 (30 min), 4. proVip2 (90 min), 5. S-tag-Vip2 (30 min), 6. S-tag-Vip2 (90 min), 7. Vip2 (30 min), 8. Vip2 (90 min). Closed arrows denote putative activated form of proVip2 proteins co-migrating with Vip2 (open arrow).

FIG. 7 shows the results of an (A) enzyme assay and (B) Western blot of engineered enzyme precursors (lanes 2 and 4) and their processed forms collected from frass of WCRW larvae (lanes 3 and 5) after 3 days post feeding. Lane: 1. MW marker, 2. proVip2, 3. proVip2 collected from frass, 4. S-tag-proVip2, 5. S-tag-proVip2 collected from frass, 6. Vip2

### BRIEF DESCRIPTION OF THE SEQUENCES IN THE SEQUENCE LISTING

SEQ ID NOs: 1-5 are oligonucleotide primers that are useful in the invention.

SEQ ID NO: 6 is the amino acid sequence of the propeptide comprised in the 4-4-12 zymogen.

SEQ ID NO: 7 is a core propeptide sequence.

SEQ ID NO: 8 shows the amino acid sequences of the propeptides comprised in the proVip2-39T and proVip2-39A zymogens.

SEQ ID NO: 9 is the amino acid sequence of the native full-length Vip2A ADP-ribosyltransferase.

SEQ ID NO: 10 is the amino acid sequence of a truncated Vip2 ADP-ribosyltransferase.

SEQ ID NO: 11 is the amino acid sequence of the 4-4-12 zymogen.

SEQ ID NO: 12 is an amino acid sequence of the proVip2-39-T and proVip2-39A zymogens.

SEQ ID NO: 13 is the nucleotide sequence of pNOV4500.

SEQ ID NO: 14 is the nucleotide sequence of pNOV4501.

SEQ ID NOs: 15-19 are amino acid sequences of insecticidal ADP-ribosyltransferases.

SEQ ID NOs: 20-23 are amino acid sequences of non-Bacillus ADP-ribosyltransferases.

### DEFINITIONS

Unless defined otherwise, all technical and scientific terms used herein have the same meaning as is commonly understood by one of ordinary skill in the art to which this invention belongs. All patents, applications, published applications and other publications and sequences from GenBank and other data bases referred to herein are incorporated by reference in their entirety. For clarity, certain terms used in the specification are defined and presented as follows.

In the context of the present invention, “corresponding to” means that when the amino acid sequences of certain proteins are aligned with a reference amino acid sequence, the amino acids that align with certain enumerated positions in the reference amino acid sequence, for example, but not limited to, a Vip2 toxin (either SEQ ID NO: 9 or SEQ ID NO: 10), but that are not necessarily in these exact numerical positions relative to the reference amino acid sequence, “correspond to” each other. An example of such an alignment is shown in Table 1. For example, the catalytic residue, E423 of Isp2a (SEQ ID NO: 18) “corresponds to” residue E428 of Vip2 (SEQ ID NO: 9), when SEQ ID NO: 9 is used as the reference amino acid sequence.

As used herein, a zymogen is an inactive or substantially inactive propeptide of a toxic protein that is activatable in a target organism or cell. A zymogen is generally larger, although not necessarily larger than the toxic protein. Zymogens may be converted to active toxins by an activator in a target organism or cell. Such an activator, for example without limitation, may be a protease or combinations of proteases which generates the mature active toxin in a target organism or cell. Thus, a zymogen of the invention is benign (having little or no detrimental effect) in a non-target organism or cell, for example a plant or plant cell or yeast cell, and is convened to a toxic protein in a target organism or cell, for example in an insect or insect cell.

As used herein, homologous means greater than or equal to 25% nucleic acid or amino acid sequence identity, typically 25% 40%, 60%, 61%, 62%, 63%, 64%, 65%, 66%, 67%, 68%, 69%, 70%, 75%, 78%, 80%, 85%, 90%, 91%, 92%, 93%, 94%, 95%, 96%, 97%, 98% or 99%; the precise percentage can be specified if necessary. For purposes herein the terms “homology” and “identity” are often used interchangeably. In general, for determination of the percentage identity, sequences are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carillo et al. (1988) SIAM J Applied Math 48:1073). By sequence identity, the numbers of conserved amino acids are determined by standard alignment algorithms programs, and are used with default gap penalties established by each supplier. Substantially homologous nucleic acid molecules would hybridize typically at moderate stringency or at high stringency all along the length of the nucleic acid of interest. Also contemplated are nucleic acid molecules that contain degenerate codons in place of codons in the hybridizing nucleic acid molecule.

The identity or homology of any nucleotide or amino acid sequence can be determined using known computer algorithms such as the “FAST A” program, using for example, the default parameters as in Pearson et al. (1988) Proc. Natl. Acad. Sci. USA 85:2444 (other programs include the GCG program package (Devereux, J., et al., Nucleic Acids Research 12(1):387 (1984)), BLASTP, BLASTN, FASTA (Atschul, S. F., et al., J Molec Biol 215:403 (1990); Guide to Huge Computers, Martin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include DNAStar “MegAlign” program (Madison, Wis.) and the University of Wisconsin Genetics Computer Group (UWG) “Gap” program (Madison Wis.). Percent homology or identity of proteins and/or nucleic acid molecules can be determined, for example, by comparing sequence information using a GAP computer program (e.g., Needleman et al. (1970) J. Mol. Biol. 48:443, as revised by Smith and Waterman ((1981) Adv. Appl. Math. 2:482). Briefly, the GAP program defines similarity as the number of aligned symbols (i.e., nucleotides or ammo acids) which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. (1986) Nucl. Acids Res. 14:6745, as described by Schwartz and Dayhoff, eds., ATLAS OF PROTEIN SEQUENCE AND STRUCTURE, National Biomedical Research Foundation, pp. 353 358 (1979); (2) a penalty of 3.0 for each gap and an additional 0.10 penalty for each symbol in each gap; and (3) no penalty for end gaps. Therefore, as used herein, the term “identity” represents a comparison between a test and a reference polypeptide or polynucleotide.

As used herein, for example, the term at least “90% identical to” refers to percent identities from 90 to 99.99 relative to the reference polypeptides. Identity at a level of 90% or more is indicative of the fact that, assuming for exemplification purposes a test and reference polynucleotide length of 100 amino acids are compared. No more than 10% (i.e., 10 out of 100) amino acids in the test polypeptide differs from that of the reference polypeptides. Similar comparisons can be made between a test and reference polynucleotides. Such differences can be represented as point mutations randomly distributed over the entire length of an amino acid sequence or they can be clustered in one or more locations of varying length up to the maximum allowable, e.g. 10/100 amino acid difference (approximately 90% identity). Differences are defined as nucleic acid or amino acid substitutions, or deletions. At the level of homologies or identities above about 85 to 90%, the result should be independent of the program and gap parameters set; such high levels of identity can be assessed readily, often without relying on software.

Another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part 1 chapter 2 “Overview of principles of hybridization and the strategy of nucleic acid probe assays” Elsevier, N.Y. Generally, highly stringent hybridization and wash conditions are selected to be about 5° C. lower than the thermal melting point (Tₘ) for the specific sequence at a defined ionic strength and pH. Typically, under “stringent conditions” a probe will hybridize to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of sets of hybridization/wash conditions that may be used to clone homologous nucleotide sequences that are substantially identical to reference nucleotide sequences of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, mM EDTA at 50° C. with washing in 1×SSC, 0.1% SDS 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

A further indication that two nucleic acid sequences or proteins are substantially identical is that the protein encoded by the first nucleic acid is immunologically cross reactive with, or specifically binds to, the protein encoded by the second nucleic acid. Thus, a protein is typically substantially identical to a second protein, for example, where the two proteins differ only by conservative substitutions.

As used herein, primer refers to an oligonucleotide containing two or more deoxyribonucleotides or ribonucleotides, generally more than three, from which synthesis of a primer extension product can be initiated. Experimental conditions conducive to synthesis include the presence of nucleoside triphosphates and an agent for polymerization and extension, such as DNA polymerase, and a suitable buffer, temperature and pH.

It is known that there is a substantial amount of redundancy in the various codons that code for specific amino acids. Therefore, this invention is also directed to those DNA sequences that contain alternative codons that code for the eventual translation of the identical amino acid. For purposes of this specification, a sequence bearing one or more replaced codons will be defined as a degenerate variation. Also included within the scope of this invention are mutations either in the DNA sequence or the translated protein that do not substantially alter the ultimate physical properties of the expressed protein. An example of such changes include substitution of an aliphatic for another aliphatic, aromatic for aromatic, acidic for another acidic, or a basic for another basic amino acid may not cause a change in functionality of the polypeptide. Also, more apparently radical substitutions may be made if the function of the residue is to maintain polypeptide solubility, including a charge reversal. It is known that DNA sequences coding for a peptide may be altered so as to code for a peptide having properties that are different than those of the naturally occurring peptide. Methods of altering the DNA sequences include, but are not limited to, site directed mutagenesis.

As used herein, toxic activity is understood to mean any action resulting in the death of a cell or a prevention of any cellular function, including but not limited to mitosis or meiosis.

“Transformation” is a process for introducing heterologous nucleic acid into a host cell or organism. In particular, “transformation” means the stable integration of a DNA molecule into the genome of an organism of interest.

“Transformed/transgenic/recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed”, “non-transgenic”, or “non-recombinant”host refers to a wild-type organism, e.g., a bacterium or plant, which does not contain the heterologous nucleic acid molecule.

Nucleotides are indicated herein by their bases by the following standard abbreviations: adenine (A), cytosine (C), thymine (T), and guanine (G). Amino acids are likewise indicated by the following standard abbreviations: alanine (Ala; A), arginine (Arg; R), asparagine (Asn; N), aspartic acid (Asp; D), cysteine (Cys; C), glutamine (Gln; Q), glutamic acid (Glu; E), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

### DETAILED DESCRIPTION

Bacterial ADP-ribosylating toxins are proteins produced by pathogenic bacteria, which are usually secreted into the extracellular medium and cause disease by altering the metabolism of eukaryotic cells (Rappuoli and Pizza, 1991). These enzymes catalyze the transfer of the ADP-ribose group from NAD to a target protein with nicotinamide release. Since actin, the major cytoskeleton forming protein in eukaryotic cells is the primary ribosylation target for Vip2 ADP-ribosyltransferase, the intracellular expression of Vip2 in plant cells could be a real challenge.

Early maize transformation experiments with Vip2 indicated that all transgenic plants had an aberrant phenotype and problems in development. Growth of transformed plants ceased at the very early developmental stage. Furthermore, experiments designed to target Vip2 protein into extra-cytoplasmic space (apoplast) did not significantly improve symptoms of plant pathology. Therefore, other approaches were needed in order to protect a plant, a non-target organism, from Vip2 toxic activity yet maintain the toxic activity in a target organism or cell, for example insects.

It is revealed here that it is possible to design novel zymogens of toxic proteins that are benign in a non-target organism or cell and that become toxic only when acted upon by an activator in a target organism or cell. It is also taught here that protein re-engineering can include altering the C-terminus or N-terminus of native toxic proteins without necessarily making the toxic protein inactive. Based on these teachings, it is now possible to design specific zymogens to be active only in target organisms or cells, while still retaining the ability to perform proper biological activity.

In one embodiment, the present invention encompasses an engineered zymogen of a toxic protein having a polypeptide chain extension fused to a C-terminus or a N-terminus of the toxic protein, wherein the zymogen is benign in a non-target organism or cell and wherein the zymogen is converted to a toxic protein when the zymogen is in a target organism or cell.

In another embodiment, the present invention encompasses an engineered zymogen of a toxic protein, the amino acid sequence of the zymogen varied from the amino acid sequence of the toxic protein by changes which comprise (a) the addition of a polypeptide chain extending from the native carboxyl terminus or amino terminus of the toxic protein, and (b) the introduction of a new carboxyl terminus or amino terminus in the zymogen, the zymogen being capable of conversion to a toxic protein in a target organism or cell.

In yet another embodiment, the present invention encompasses a zymogen, wherein the toxic protein is an ADP-ribosyltransferase. Typically, the ADP-ribosyltransferase ribosylates actin.

In another embodiment, the present invention encompasses a zymogen, wherein the ADP-ribosyltransferase comprises an amino acid sequence with at least 69% or 78% or 85% or 93% or 95% sequence identity to SEQ ID NO:9 and wherein the ADP-ribosyltransferase has a catalytic residue that corresponds to E428 of SEQ ID NO:9 and NAD binding residues that correspond to Y307, R349, E355, F397, and R400 of SEQ ID NO:9. In one aspect of this embodiment, the ADP-ribosyltransferase is insecticidal.

In yet another embodiment, the present invention encompasses a zymogen, wherein the ADP-ribosyltransferase is a Vip2 toxin. In one aspect of this embodiment the Vip2 toxin is selected from a group consisting of SEQ ID NO: 9, 10, 15, 16, 17, 18, and 19.

In still another embodiment, the present invention encompasses a zymogen, wherein the polypeptide extension comprises an amino acid sequence of at least 21 residues long and having a tryptophan (Trp; W) residue at position 3, 14, and 19. In one aspect of this embodiment the polypeptide extension comprises SEQ ID NO:6.

In another embodiment, the present invention encompasses a zymogen, wherein the polypeptide extension comprises SEQ ID NO:8.

The present invention further encompasses a zymogen of an ADP-ribosyltransferase wherein the polypeptide chain extension is fused to the C-terminus of the ADP-ribosyltransferase.

In another embodiment, the present invention encompasses a zymogen, wherein the non-target organism or cell is a plant, a plant cell, or a yeast cell. In one aspect of this embodiment, the plant or plant cell is selected from the group consisting of sorghum, wheat, tomato, cole crops, cotton, rice, soybean, sugar beet, sugarcane, tobacco, barley, oilseed rape, and maize. In another aspect of this embodiment the yeast cell is Saccharomyces cerevisae.

In yet another embodiment, the present invention encompasses a zymogen comprising SEQ ID NO:11 or SEQ ID NO:12.

In still another embodiment, the present invention encompasses an isolated nucleic acid molecule comprising a nucleotide sequence that encodes a zymogen of the invention; a recombinant vector comprising the nucleic acid molecule and a yeast cell comprising the recombinant vector.

In another embodiment, the present invention encompasses transgenic plants comprising a zymogen of the invention.

In still, another embodiment, the present invention encompasses a method of making a zymogen of a toxic protein, the method comprising the steps of: (a) designing a polypeptide chain which extends from a terminus of the toxic protein; (b) making a library of expression plasmids which will express a precursor including the polypeptide chain upon transformation into a host organism or cell; (c) expressing the precursors in a genetic system that is naturally susceptible to the toxic protein; (d) recovering cells of the genetic system which survive step (c); (e) isolating the precursors from the cells of step (d); (f) testing the precursors for biological activity against a target organism or cell; and (g) identifying the biologically active precursors as zymogens.

In another embodiment, the present invention encompasses a genetic system that allows for efficient identification of zymogen precursors of toxic proteins, wherein the toxic protein in the precursor is inactive or substantially inactive and wherein the zymogen is benign in a non-target host organism or cell and is converted to a toxic protein when the zymogen is in a target organism or cell.

In yet a further embodiment, pharmaceutical compositions containing the novel zymogens of the invention are encompassed by the present invention. Such pharmaceutical compositions should have efficacy as for example, anti-cancer agents.

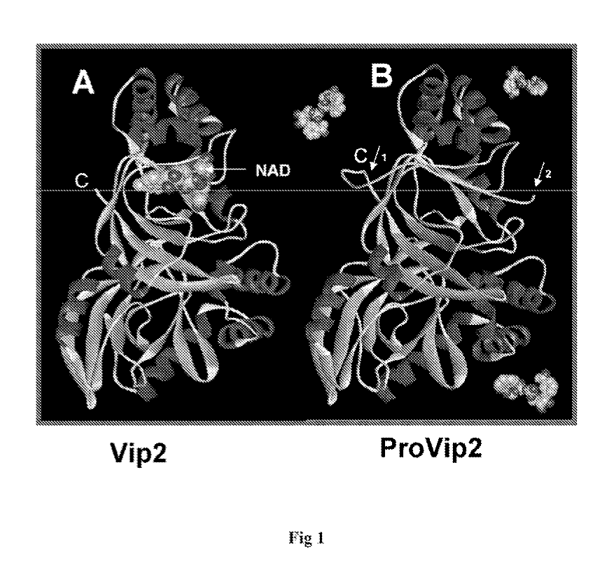

In one embodiment of the present invention methods are disclosed to create a zymogen of Vip2 ADP-ribosyltransferase for reducing phytotoxicity when expressed in planta. As Vip2 ribosylates one of the most conserved proteins in nature, it is reasonable to assume that this toxin would likely be toxic to any cells requiring actin for their viability. In its native form, expression of Vip2 protein in plants is lethal and thus can not be used for transgenic purposes. However, an engineered zymogen would need to be activated by the digestive proteases of a target pest in order to exert its lethal function. The proper extension of a polypeptide chain from a terminus of a Vip2 ADP-ribosyltransferase may, without limitation, interfere with its enzymatic function by four mechanisms: 1) steric blocking of the active site, 2) interference with the NAD-binding site, 3) imparting a change in enzyme conformation, or 4) introducing a decrease in overall protein stability. Since the C-terminal end of Vip2 is in closer proximity to the functional sites of the protein than the N-terminus (FIG. 1), it was envisioned that the addition of a polypeptide chain extension at the C-terminal part of the protein might have a better chance to mask Vip2 enzymatic activity. In order to find functional propeptide sequences, a genetic system that would efficiently select for Vip2 zymogen precursors with suppressed enzymatic function had to be designed.

Disclosed herein is an in vivo genetic system for selection of defective Vip2 variants in yeast. Using random elongation mutagenesis at the C-terminus of the protein and selection in yeast, a Vip2 proenzyme was identified with significantly reduced enzymatic activity which was benign to corn plants thus causing no developmental pathology under greenhouse conditions. Moreover, the engineered zymogen is still powerful enough to cause rootworm mortality due to activation by proteases in the corn rootworm digestive system to a wild type enzymatic form.

Using this disclosure, one skilled in the art can easily adopt the genetic system for rapid screening to determine potential functional significance of amino acid residues in any ADP-ribosyltransferase, particularly an actin ADP-ribosyltransferase, and for identifying these critical residues. Vip2 shares significant sequence similarity with enzymatic components of other insecticidal and non-insecticidal toxins, including those listed below in Table 1 and Table 2, respectively. These Vip2-like ADP-ribosyltransferases have several structural features in common that relate to their function. These key structural features that are important in the biological activity of Vip2-like ADP-ribosyltransferases include the catalytic residue corresponding to E428 of Vip2 (SEQ ID NO: 9), the NAD binding residues corresponding to Y307, R349, E355, F397 and R400 of Vip2 (SEQ ID NO: 9), the “STS motif” corresponding to residues 386-388 of Vip2 (SEQ ID NO: 9), that stabilizes the NAD binding pocket, and the NAD binding pocket formed by residues corresponding to E426 and E428 of Vip2 (SEQ ID NO: 9). Therefore, a zymogen may be designed for any ADP-ribosyltransferase that has a similar structure/function relationship to Vip2, whereby the zymogen is benign in a non-target organism or cell but active in a target organism or cell. Table 1 shows an alignment of insecticidal toxins that have homology to Vip2. Table 2 shows an alignment of non-insecticidal toxins that have homology to Vip2. Each of these ADP-ribosyltransferases (SEQ ID NOs 15-19 of Table 1 and SEQ ID NOs 20-23 of Table 2) have a catalytic residue, NAD binding residues, an STS motif and NAD binding pocket residues that correspond to those residues of Vip2 (SEQ ID NO:9).

TABLE 1
Homologous ADP-ribosylating toxins.

| | Sequence | Start | End | Length | % Identity |
|---|---|---|---|---|---|
| Ref 1 | Vip2Aa (SEQ ID NO: 9) | 1 | 462 | 462 aa | |
| 2 | Vip2Ac (SEQ ID NO: 15) | 1 | 462 | 462 aa | 95 |
| 3 | Vip2A-BR (SEQ ID NO: 16) | 1 | 462 | 462 aa | 93 |
| 4 | ACH42759 (SEQ ID NO: 17) | 1 | 462 | 462 aa | 85 |
| 5 | Isp2a (SEQ ID NO: 18) | 1 | 457 | 457 aa | 78 |
| 6 | Isp2b (SEQ ID NO: 19) | 1 | 460 | 460 aa | 69 |

| | | |
|---|---|---|
| Vip2Aa | 1 | MKRMEGKLFMVSKKLQVVTKTVLLSTVFSISLLNNEVIKAEQLNINSQSKYTNLQNLKIT |
| Vip2Ac | 1 | MKRMEGKLFMVSTKLQAVTKAVLLSTVLSISLLNNEVIKAEQLNMNSQNKYTNFENLKIT |
| Vip2A-BR | 1 | MQRMEGKLFMVSKKLQAVTKTVLLSTVLSISLLNNEEVKAEQLNINSQNKYTNFQNLKIT |
| ACH42759 | 1 | MKRMEGKLFMVSRKLQLVTKALLFSTVLSIPLLNNEEVKAEHLNLNSQSKYPSFQNQKIT |
| Isp2a | 1 | ---MIVIIFTNVKGGNELKKNFYKNLICMSALLLAMPISSNVTYAYGSEKVDYL--VKTT |
| Isp2b | 1 | MKRMEERLFMVSKKLQLITKTLVFSTVLSIPLLNNSEIKAEQLNMNSQIKYPNFQNINIA |
| Vip2Aa | 61 | DKVEDFKEDKEKAKEWGKEKEKEWKLTATEKGKMNNFLDNKNDIKTNYKEITFSMAGSFE |
| Vip2Ac | 61 | DKVEDFKEDKEKAKEWGKEKEKEWKLTATEKGKMNNFLDNKNDIKTNYKEITFSMAGSFE |
| Vip2A-BR | 61 | DNAEDFKEDKEKAKEWGEEKEKEWKLTATEKGKMNNFLDNKNDIKTNYKEITFSMAGSFE |
| ACH42759 | 61 | DNAEDFKEDKEKAKEWGEVKEKEWKLTATEKRKINDFLNDTNKIKTNYKEITFSMAGSFE |
| Isp2a | 56 | NNTEDFKEDKEKAKEWGKEKEKEWKLTVTEKTRMNNFLDNKNDIKKNYKEITFSMAGSFE |
| Isp2b | 61 | DKPVDFKEDKEKAREWGKEKEKEWKLTATEKGKINDFLDDKDGLKTKYKEINFSKNFEYE |
| Vip2Aa | 121 | DEIKDLKEIDKMFDKTNLSNSIITYKNVEPITIGFNKSLTEGNTINSDAMAQFKEQFLDR |
| Vip2Ac | 121 | DEIKDLKEIDKIFDKANLSSPIITYKNVEPATIGFNKSLTEGNTINSDAMAQFKEQFLDR |
| Vip2A-BR | 121 | DEIKDLKEIDKIFDKANLSSSIITYKNVEPATIGFNKSLTEGNTINSDAMAQFKEQFLGK |
| ACH42759 | 121 | DELKDLKEIDKMFDKANLSSSIITYKNVEPATIGFNKSLTEGNTINSDVMAQFKEQFLGK |
| Isp2a | 116 | DEIKDLKEIDKMFDKANLSSSIVTYKNVEPSTIGFNKPLTEGNTINTDVQAQFKEQFLGK |
| Isp2b | 121 | TELKELEKINTMLDKANLTNSIVTYKNVEPTTIGFNQSLIEGNQINAEAQQKFKEQFLGQ |
| Vip2Aa | 181 | DIKFDSYLDTHLTAQQVSSKERVILKVTVPSGKGSTTPTKAGVILNNSEYKMLIDNGYMV |
| Vip2Ac | 181 | DIKFDSYLDTHLTVQQVSSKERVILKVKVPSGKGSTTPTKAGIILNNSEYKMLIDNGYMV |
| Vip2A-BR | 181 | DMKFDSYLDTHLTAHQVSSKKRVILKVTVPSGKGSTTPTKAGVILTNNEYKMLIDNGYVL |
| ACH42759 | 181 | DIKFDSYLDTHLTVQQVSSKERVILKVTVPSGKGSTNPTKAGVILDGNEPKMLIDNGYVL |
| Isp2a | 176 | DIKFDSYLDTHLTAQNVSSKERIILQVTVPSGKGSTIPTKAGVILNNNEYKMLIDNGYVL |
| Isp2b | 181 | DIKFDSYLDMHLTEQNVSSKERVILKVTVPSGKGS-TPTKAGVVLNNNEYKMLIDNGYVL |
| Vip2Aa | 241 | HVDKVSKVVKKGVECLQIEGTLKKSLDFKNDINAEAHSWGMKNYEEWAKDLTDSQREALD |
| Vip2Ac | 241 | HVDKVSKVVKKGVECLQVEGTLKKSLDFKNDINAGAHSWGMKNYEEWAKDLTDLQREALD |
| Vip2A-BR | 241 | HVDKVSKVVKKGMECLQVEGTLKKSLDFKNDINAEAHSWGMKIYEDWAKNLTASQREALD |
| ACH42759 | 241 | HVDKVSKVVKKGLECLQVEGTLKKSLDFKNDISAKAHSWGMKNYEEWAANLTDSQRKALD |
| Isp2a | 236 | HVDNISKVVKKGYECLQIQGTLKKSLDFKNDINAEAHRWGMKNYEGWAKNLTDPQREALD |
| Isp2b | 240 | HVENITKVVKKGQECLQVEGTLKKSLDFKNDSDGKGDSWGKKNYKEWSDTLTTDQRKDLN |
| | | ↓ ↓ ↓ |
| Vip2Aa | 301 | GYARQDYKEINNYLRNQGGSGNEKLDAQIKNISDALGKKPIPENITVYRWCGMPEFGYQI |
| Vip2Ac | 301 | GYARQDYKEINNYLRNQGGNGNEKLDAQIKNISDALGKKPIPENITVYRWCGMPEFGYQI |
| Vip2A-BR | 301 | GYARQDYKEINNYLRNQGGSGNEKLDAQIKNISDALGKKPIPENITVYRWCGMPEFGYQI |
| ACH42759 | 301 | GYARQDYKKINDYLRNQGGSGNEQLDAQIKNISETLNNKPIPENITVYRWCGMPEFGYQI |
| Isp2a | 296 | GYARQDYKQINDYLRNQGGSGNEKLDTQIKNISEALEKQPIPENITVYRWCGMAEFGYQI |
| Isp2b | 300 | DYGARGYTEINKYLR-EGGTGNTELEEKIKNISDALEKNPIPENITVYRYCGMAEFGYPI |
| | | ↓ ↓ |
| Vip2Aa | 361 | SDPLPSLKDFEEQFLNTIKEDKGYMSTSLSSERLAAFGSRKIILRLQVPKGSTGAYLSAI |
| Vip2Ac | 361 | SDPLPSLKDFEEQFLNTIKEDKGYMSTSLSSERLAAFGSRKIILRLQVPKGSTGAYLSAI |
| Vip2A-BR | 361 | SDPLPSLKDFEEQFLNTIKEDKGYMSTSLSSERLAAFGSRKIILRLQVPKGSTGAYLSAI |
| ACH42759 | 361 | SEPLPALKDFEWEFLNTIKEDKGYISTSLSSERLAAFGSRKIILRLQIPKGSKGAYLSAI |
| Isp2a | 356 | SDPLPSLKEMEEKFLNTMKEDKGYMSTSLSSERLSAFGSRKFILRLQVPKGSTGAYLSAI |
| Isp2b | 359 | KPEAPSVQDFEERFLDTIKEEKGYMSTSLSSDA-TAFGARKIILRLQVPKGSSGAYVAGL |
| | | ♦ |
| Vip2Aa | 421 | GGFA-SEKEILLDKDSKYHIDKVTEVIIKGVKRYVVDATLLTN |
| Vip2Ac | 421 | GGFA-NEKEILLDKDSKYHIDKVTEVIIKGVKRYVVDATLLTN |
| Vip2A-BR | 421 | GGFA-SEKEILLDKDSKYHIDKATEVIIKGVKRYVVDATLLTN |
| ACH42759 | 421 | GGFA-NEKEILLDKDSKYHINKITEVVIKGIKRYVVDATLLTN |
| Isp2a | 416 | GGFA-SEKEILIDKDSNYHIDKITEVVIKGVKRYVVDATLLTK |
| Isp2b | 418 | DGFKPAEKEILIDKGSKYRIDKVTEVVVKGTRKLVVDATLLTK |

The catalytic residue, Glu428 in Vip2, is marked with a ♦ above the sequences.
Residues involved in NAD binding are indicated with a ↓.
The STS motif is underlined.

TABLE 2
Homologous ADP-ribosylating toxins.

| | Sequence | Start | End | Length | % Identity |
|---|---|---|---|---|---|
| Ref 1 | Vip2Aa (SEQ ID NO: 9) | 1 | 462 | 462 aa | |
| 2 | Cd-Cdta toxin (SEQ ID NO: 20) | 1 | 463 | 463 aa | 31 |
| 3 | Cp-IotaA chain (SEQ ID NO: 21) | 1 | 454 | 454 aa | 29 |
| 4 | Cs-Sa toxin (SEQ ID NO: 22) | 1 | 459 | 459 aa | 29 |
| 5 | CbC2 toxin (SEQ ID NO: 23) | 1 | 431 | 431 aa | 26 |

| | | |
|---|---|---|
| Vip2Aa | 1 | MKRMEGKLFMVSKKLQVVTKTVLLSTVFSISLLNNEVIKAEQLNINSQSKYTNLQNLKI- |
| Cd-CdtA toxi | 1 | MKK-----FRKHKRISNCISILLILYLTLGGLLPNN-IYAQDLQSYSE-KVCNTTYKAP- |
| Cp-IotaA cha | 1 | MKK-------VNKSISVFLILYLILT-------------------SSFPSYTYAQDLQIA |
| Cs-Sa toxin | 1 | MKKYKNNCISILLMLFLILTGLFPNTVFAQG--------AQSYDFRT---INNIANYSA- |
| CbC2 toxin | 1 | ----------------------------------MPIIK--------------------- |
| Vip2Aa | 60 | ----TDKV-----EDFKEDKEKAKEWG-KEKEK--EW--KLTATEKGKMNNFL--DNKND |
| Cd-CdtA toxi | 53 | ----IERP-----EDFLKDKEKAKEWERKEAERI-EQ--KLERSEKEALESY----KKDS |
| Cp-IotaA cha | 35 | SNYITDRAFIERPEDFLKDKENAIQWE-KKEAERVEK--NLDTLEKEALELYK--KDSEQ |
| Cs-Sa toxin | 49 | ----IERP-----EDFLKDKEKAKDWERKEAERI-EK--NLEKSEREALESYK--KDAVE |
| CbC2 toxin | 6 | -----EPI-----DFINKPESEAQKWG-KEEEK--RWFTKLNNLEEVAVNQLKTKEDKTK |
| Vip2Aa | 104 | IKTNY---KEIT---FSMAGSFE----DEI----KDLKEIDKMFD---KTNLSNSIITYK |
| Cd-CdtA toxi | 97 | VEISK---YSQT---RNYFYDYQ----IEANSREKEYKELRNAIS---KNKIDKPMYVYY |
| Cp-IotaA cha | 90 | I-SNYSQTRQYF---YDYQIESN----PRE----KEYKNLRNAIS---KNKIDKPINVYY |
| Cs-Sa toxin | 95 | I-SKY---SQVRNYFYDYPIEAN----TRE----KEYKELKNAVS---KNKIDKPMYVYY |
| CbC2 toxin | 53 | IDNFS---TDIL---FSSLTAIEIMKEDEN----QNLFDVERIREALLKNTLDREVIGYV |
| Vip2Aa | 147 | NVEPTTIGFNKSL-T--E---G-NTINSDAMAQFKEQFLDRDIKFDSYLDTHLTAQQVSS |
| Cd-CdtA toxi | 144 | FESPEKFAFNKVIRT--E---NQNEISLEKFNEFKETIQNKLFKQDGFKDISLYEPGKGD |
| Cp-IotaA cha | 135 | FESPEKFAFNKEIRT--E---NQNEISLEKFNELKETIQDKLFKQDGFKDVSLYEPGNGD |
| Cs-Sa toxin | 140 | FESPEKFAFNKEI-RAES---Q-NEISLERFNEFKATIQDKLFKQDGFKDISLYEPGNGD |
| CbC2 toxin | 103 | NFTPKELGINFSI-R--DVELN-RDISDEILDKVRQQIINQEYTKFSFVSLGLNDNSIDE |
| Vip2Aa | 200 | KER--VILKVTVPSGKGSTTPTKAGVI--LNNSEYKMLIDNGYMVHVDKVSKVVKKGVEC |
| Cd-CdtA toxi | 199 | EKPTPLLMHLKLPRNTGMLPYT--------NTNNVSTLIEQGYSIKIDKIVRIVIDGKHY |
| Cp-IotaA cha | 190 | EKP--TPLLIHLK------LPKNTGMLPYINSNDVKTLIEQDYSIKIDKIVRIVIEGKQY |
| Cs-Sa toxin | 195 | KKS--TPLLIHLK------LPKDTGMLPYSNSNDVSTLIEQGYSIKIDKIVRIVLEGKQY |
| CbC2 toxin | 159 | SIP--VIVKTRVP------TTFNYGVL--NNKETVSLLLNQGFSIIPESAIITTIKGKDY |
| | | ↓ |
| Vip2Aa | 256 | LQIEGTLKKSLDFKNDINAEAHSWGMKNYEEWAKDLTDSQREALDGYARQDYKEINNYLR |
| Cd-CdtA toxi | 251 | IKAEASVVSSLDFKDDV-SKGDSWGKANYNDWANKLTPNELADVNDYMRGGYTAINNYLI |
| Cp-IotaA cha | 242 | IKAEASIVNSLDFKDDV-SKGDLWGKENYSDWSNKLTPNELADVNDYMRGGYTAINNYLI |
| Cs-Sa toxin | 247 | IKAEASVVSCLDFKDDV-SKGDSWGKANYSDWSNKLSSDELAGVNDYMRGRYTAINNYLI |
| CbC2 toxin | 209 | ILIEGSLSQELDF---YNKGSEAWGEKNYGDYVSKLSQEQLGALEGYLHSDYKAINSYLR |
| | | ↓ ↓ |
| Vip2Aa | 316 | NQG--GSGNE--KLDAQIKNISDALGKKPIPENITVYRWC-GMP------------EFGY |
| Cd-CdtA toxi | 310 | SNGPVNNPNP--ELDSKITNIENALKREPIPTNLTVYRRS-GPQ------------EFGL |
| Cp-IotaA cha | 301 | SNGPLNNPNP--ELDSKVNNIENALKLTPIPSNLIVYRRS-GPQ------------EFGL |
| Cs-Sa toxin | 306 | ANG--PTNNPNAELDAKINNIENALKREPIPANLVVYRRS-GPQ------------EFGL |
| CbC2 toxin | 266 | NNR--VPNND--ELNKKIELISSALSVKPIPETLIAYRRVDGIPFDLPSDFSFDKKENGE |
| | | ↓ ↓ |
| Vip2Aa | 359 | QISDP------LPSLKDFEEQFLNTIKEDKGYMSTSLSSERLAAFGSRKIILRLQVPKGS |
| Cd-CdtA toxi | 355 | TLTSPEYDFNKLENIDAFKSKWEGQALSYPNFISTSIGSVNMSAFAKRKIVLRITIPKGS |
| Cp-IotaA cha | 346 | TLTSPEYDFNKIENIDAFKEKWEGKVITYPNFISTSIGSVNMSAFAKRKIILRINIPKDS |
| Cs-Sa toxin | 351 | TLSSPEYDFNKVENIDAFKEKWEGQTLSYPNFVSTSIGSVNMSAFAKRKIVLRISIPKNS |
| CbC2 toxin | 322 | IIADK------T-KLNEFIDKWTGKEIENLSFSSTSLKSTPLS-FSKSRFIFRLRLSEGT |
| | | ♦ |
| Vip2Aa | 413 | TGAYLSAIGGFASEKEILLDKDSKYHIDKVTEV--IIKGVKRY---VVDATLLTN--- |
| Cd-CdtA toxi | 415 | PGAYLSAIPGYAGEYEVLLNHGSKFKINKISDY--KDGTITKL---IVDATLIP---- |
| Cp-IotaA cha | 406 | PGAYLSAIPGYAGEYEVLLNHGSKFKINKVDSY--KDGTVTKL---ILDATLIN---- |
| Cs-Sa toxin | 411 | PGAYLSAIPGYAGEYEVLLNHGSKFKISKIDSY--KDGTTTKL---IVDRTLID---- |
| CbC2 toxin | 374 | IGAFIYGFSGFQDEQEILLNKNSTFKIFRITPITSIINRVTKMTQVVIDAEVIQNKEI |

The catalytic residue, Glu428 in Vip2, is marked with a ♦ above the sequences.
Residues involved in NAD binding are indicated with a ↓.
The STS motif is underlined.

In another embodiment, at least one of the insecticidal toxins of the invention is expressed in a higher organism, e.g., a plant. In this case, transgenic plants expressing effective amounts of the zymogens protect themselves from insect pests. When the insect starts feeding on such a transgenic plant, it also ingests the expressed zymogen. The zymogen is activated in the target insect and this will deter the insect from further biting into the plant tissue or may even harm or kill the insect. A nucleotide sequence of the present invention is inserted into an expression cassette, which is then preferably stably integrated in the genome of the plant. Plants transformed in accordance with the present invention may be monocots or dicots and include, but are not limited to, maize, wheat, barley, rye, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugar beet, sunflower, rapeseed, clover, tobacco, carrot, cotton, alfalfa, rice, potato, eggplant, cucumber, Arabidopsis, and woody plants such as coniferous and deciduous trees.

Once a desired nucleotide sequence has been transformed into a particular plant species, it may be propagated in that species or moved into other varieties of the same species, particularly including commercial varieties, using traditional breeding techniques.

A nucleotide sequence of this invention is preferably expressed in transgenic plants, thus causing the biosynthesis of the corresponding toxin in the transgenic plants. In this way, transgenic plants with enhanced resistance to insects are generated. For their expression in transgenic plants, the nucleotide sequences of the invention may require modification and optimization. Although in many cases genes from microbial organisms can be expressed in plants at high levels without modification, low expression in transgenic plants may result from microbial nucleotide sequences having codons that are not preferred in plants. It is known in the art that all organisms have specific preferences for codon usage, and the codons of the nucleotide sequences described in this invention can be changed to conform with plant preferences, while maintaining the amino acids encoded thereby. Furthermore, high expression in plants is best achieved from coding sequences that have at least about 35% GC content, preferably more than about 45%, more preferably more than about 50%, and most preferably more than about 60%. Microbial nucleotide sequences that have low GC contents may express poorly in plants due to the existence of ATTTA motifs that may destabilize messages, and AATAAA motifs that may cause inappropriate polyadenylation. Although preferred gene sequences may be adequately expressed in both monocotyledonous and dicotyledonous plant species, sequences can be modified to account for the specific codon preferences and GC content preferences of monocotyledons or dicotyledons as these preferences have been shown to differ (Murray et al. Nucl. Acids Res. 17:477-498 (1989)). In addition, the nucleotide sequences are screened for the existence of illegitimate splice sites that may cause message truncation. All changes required to be made within the nucleotide sequences such as those described above are made using well known techniques of site directed mutagenesis, PCR, and synthetic gene construction using the methods described in the published patent applications EP 0 385 962, EP 0 359 4721, and WO 93/07278.

The present invention also encompasses recombinant vectors comprising the nucleic acid sequences of this invention. In such vectors, the nucleic acid sequences are preferably comprised in expression cassettes comprising regulatory elements for expression of the nucleotide sequences in a transgenic host cell capable of expressing the nucleotide sequences. Such regulatory elements usually comprise promoter and termination signals and preferably also comprise elements allowing efficient translation of polypeptides encoded by the nucleic acid sequences of the present invention. Vectors comprising the nucleic acid sequences are usually capable of replication in particular host cells, preferably as extrachromosomal molecules, and are therefore used to amplify the nucleic acid sequences of this invention in the host cells. In one embodiment, non-target organisms or cells for such vectors are microorganisms, such as bacteria, in particular Agrobacterium. In another embodiment, a non-target organism or cell for such vectors is a eukaryotic cell, such as a yeast cell, a plant, or a plant cell. In still another embodiment, a plant or plant cell comprises a maize plant or maize cell. Recombinant vectors are also used for transformation of the nucleotide sequences of this invention into transgenic host cells, whereby the nucleotide sequences are stably integrated into the DNA of such transgenic host cells. In one embodiment, such transgenic host cells are eukaryotic such as yeast cells, insect cells, or plant cells. In another embodiment, the transgenic host cells are plant cells, such as maize cells.

In one embodiment of the present invention, a nucleotide sequence of the invention is directly transformed into the non-target organism or cell genome. For Agrobacterium-mediated transformation, binary vectors or vectors carrying at least one T-DNA border sequence are suitable, whereas for direct gene transfer any vector is suitable and linear DNA containing only the construction of interest may be preferred. In the case of direct gene transfer, transformation with a single DNA species or co-transformation can be used (Schocher et al. Biotechnology 4:1093-1096 (1986)). For both direct gene transfer and Agrobacterium-mediated transfer, transformation is usually (but not necessarily) undertaken with a selectable marker that may provide resistance to an antibiotic (kanamycin, hygromycin or methotrexate) or a herbicide (basta). Plant transformation vectors comprising a nucleic acid sequence encoding a zymogen of the present invention may also comprise genes (e.g. phosphomannose isomerase; PMI) which provide for positive selection of the transgenic plants as disclosed in U.S. Pat. Nos. 5,767,378 and 5,994,629, herein incorporated by reference. The choice of selectable marker is not, however, critical to the invention.

In another embodiment of the present invention, a nucleotide sequence of the invention is directly transformed into the plastid genome. A major advantage of plastid transformation is that plastids are generally capable of expressing bacterial genes without substantial codon optimization, and plastids are capable of expressing multiple open reading frames under control of a single promoter. Plastid transformation technology is extensively described in U.S. Pat. Nos. 5,451,513, 5,545,817, and 5,545,818, in PCT application no. WO 95/16783, and in McBride et al. (1994) Proc. Natl. Acad. Sci. USA 91, 7301-7305.-The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab, Z., Hajdukiewicz, P., and Maliga, P. (1990) Proc. Natl. Acad. Sci. USA 87, 8526-8530; Staub, J. M., and Maliga, P. (1992) Plant Cell 4, 39-45). This resulted in stable homoplasmic transformants at a frequency of approximately one per 100 bombardments of target leaves. The presence of cloning sites between these markers allowed creation of a plastid targeting vector for introduction of foreign genes (Staub, J. M., and Maliga, P. (1993) EMBO J. 12, 601-606). Substantial increases in transformation frequency are obtained by replacement of the recessive rRNA or r-protein antibiotic resistance genes with a dominant selectable marker, the bacterial aadA gene encoding the spectinomycin-cletoxifying enzyme aminoglycoside-3′-adenyltransferase (Svab, Z., and Maliga, P. (1993) Proc. Natl. Acad. Sci. USA 90, 913-917). Previously, this marker had been used successfully for high-frequency transformation of the plastid genome of the green alga Chlamydomonas reinhardtii (Goldschmidt-Clermont, M. (1991) Nucl. Acids Res. 19:4083-4089). Other selectable markers useful for plastid transformation are known in the art and encompassed within the scope of the invention. Typically, approximately 15-20 cell division cycles following transformation are required to reach a homoplastidic state. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% of the total soluble plant protein. In one embodiment of this invention, a nucleotide sequence of the present invention is inserted into a plastid-targeting vector and transformed into the plastid genome of a desired plant host. Plants homoplastic for plastid genomes containing a nucleotide sequence of the present invention are obtained, and are preferentially capable of high expression of the nucleotide sequence.

Plainkum et al (2003) reported the creation of a zymogen from ribonuclease A by circular permutation and introduction of a highly specific protease site into a short peptide linking the N and C termini. In the case of Vip2 ADP-ribosyltransferase and other ADP-ribosyltransferases, the N and C termini are too far apart making it difficult to circularly-permutate its polypeptide chain with a short peptide. Moreover, once eaten by a target insect pest, an engineered Vip2 zymogen will be exposed to a whole set of proteolytic enzymes in the digestive system. Accordingly, a Vip2 zymogen has to be at least marginally stable and activatable in this harsh environment in order to impart toxicity. Due to the complexity of the problem, the strategy disclosed herein relied on an engineering approach for zymogen design, involving random extension of a C-terminal polypeptide chain and selection in yeast. The selected proenzyme proved to be benign in transgenic plants under greenhouse conditions and can be processed and activated in vivo by plant pest digestive proteases. The present invention thus represents the first example of applying the protein engineering approach for zymogen creation of an ADP-ribosylating toxin and provides a teaching of a more general strategy for solving certain challenges of using toxic proteins in biotechnology research and applications.

### EXAMPLES

The invention will be further described by reference to the following detailed examples. These examples are provided for the purposes of illustration only, and are not intended to be limiting unless otherwise specified. Standard recombinant DNA and molecular cloning techniques used here are well known in the art and are described by J. Sambrook, et al., Molecular Cloning: A Laboratory Manual, 3d Ed., Cold Spring Harbor, N.Y.: Cold Spring Harbor Laboratory Press (2001); by T. J. Silhavy, M. L. Berman, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, New York, John Wiley and Sons Inc., (1988), Reiter, et al., Methods in Arabidopsis Research, World Scientific Press (1992), and Schultz et al., Plant Molecular Biology Manual, Kluwer Academic Publishers (1998).

### Example 1

### Microbial Strains, Plasmids and Expression Constructs

Escherichia coli strain DH5α was used for routine cloning experiments. Proteins were expressed in Escherichia coli strain BL21-Gold (DE3) (Stratagene; La Jolla, Calif.). For yeast transformation, a strain of Saccharomyces cerevisae INVSc1 (Invitrogen; Carlsbad, Calif.) was used. Two commercially available yeast expression vectors, the high-copy number pYES2 (Invitrogen, Carlsbad, Calif.) and a low-copy number p416GALS (ATCC, Manassas, Va.), were used for inducible protein expression in Saccharomyces cerevisae. These plasmids are shuffle vectors and can be propagated both in Escherichia coli and Saccharomyces cerevisae.

A synthetic, maize optimized vip2 gene (Warren et al., 2000) coding for the mature form of a Vip2 protein was introduced into the yeast expression vector pYES2 with a BamHI-EcoRI cassette, producing the plasmid pMJ1. In addition, during subcloning from the original source vector, two other genetic elements located downstream of the vip2 gene, inverted intron #9 from maize phosphoenolpyruvate carboxylase gene (Matsuoka and Minami, 1989) and a 35S transcription terminator from cauliflower mosaic virus (Pietrzak et al., 1986) were included in the subcloned BamHI-EcoRI fragment. The mature secreted form of Vip2 protein from Bacillus cereus presumably starts with amino acid Leu54 (Warren et al., 2004). For the work reported herein a Vip2 protein which retains this exact sequence was used and is disclosed as SEQ ID NO: 10. In order to attach propeptide sequences to the Vip2 protein, a unique AatII site was engineered at the end of the vip2 gene by replacing the last codon AAC (Asn) with TCC (Ser) (SEQ ID NO: 10). Since the last amino acid substitution (N462S) does not affect Vip2 toxicity in yeast, this protein/gene variant was designated as a wild-type (“wt”) (wtVip2 protein or wtvip2 gene). A high-copy yeast expression plasmid carrying the wtVip2 gene in pYES2 backbone was designated pMJ5 and a p416GALS-based low-copy number version with the wtvip2 gene was designated pMJ7.

For protein production in Escherichia coli, expression constructs in a pET29a system (Novagen, Madison, Wis.) were prepared. pMJ23 expression plasmid has the wtvip2 gene inserted in pET29a via SacI-XhoI sites, providing expression of Vip2 protein with a N-terminally attached S-tag. Plasmid constructs expressing the S-tag version of engineered Vip2 proenzymes (pMJ24, pMJ25) were prepared by introducing proVip2 genes into pET29a via SacI-XhoI sites. For expression of proteins without the S-tag, coding regions of polypeptides were amplified by PCR and inserted via NdeI-XhoI sites into pET29a. For PCR amplification of wtvip2 gene the following set of oligonucleotides were used; MJ109 (forward): 5′-TATACATATGCTGCAGAACCTGAAGATCACC-3′ (SEQ ID NO: 1) and MJ111 (reverse): 5′-TCTAGATGCATGCTCGAGCTAGGACGTCAGCAGGGT-3′ (SEQ ID NO: 2). For amplification of proVip2 gene, the MJ109 forward primer was used in combination with MJ113: 5′-TCTAGATGCATGCTCGAGTCACTTCACTTCACTGTA-3′ (SEQ ID NO: 3). Assembled expression constructs without S-tag sequence were designated pMJ72 (“wt” Vip2 in pET29a) and pMJ73 (proVip2 in pET29a).

### Example 2

### Preparation of a Propeptide Library by Random Elongation Mutagenesis

Randomized codons were incorporated into a synthetic oligonucleotide that was used as a forward primer for PCR amplification of the region localized downstream of the vip2 gene. An NNS triplet was used for complete codon randomization, where N represents equal amount (25%) of each nucleotide, A, G, C and T, and S is 50% each G and C. The reverse oligonucleotide initiated DNA synthesis from the plasmid backbone. In the first round of mutagenesis, a stretch of 21 codons were completely randomized. The strategy was to generate a proenzyme molecule (proVip2) that preserved amino acids deemed critical to survive in yeast as determined during initial selection. In order to attach a propeptide library to the C-terminal end of Vip2 ADP-ribosyltransferase, a recognition site for AatII restriction endonuclease was created at the end of the vip2 gene. This modification changes the last amino acid of Vip2 into serine (N462S), without compromising toxicity in yeast. Therefore, this mutant was designated as “wt” Vip2 (equivalent to native Vip2). A library encoding for random peptides (21-mers) was attached, via the engineered AatII site, to the 3′ end of the vip2 gene in the yeast low-copy copy number plasmid pMJ7 (p416GALS backbone).

In the second round of mutagenesis, 7 out of 21 amino acids preselected in the first round of mutagenesis were then randomized. The following synthetic oligonucleotides were used for randomizing of seven positions: 5′GATCAGGGACGTCCGTAGGATGGGTA(NNS)₃GGTGAAGTATTC(NNS)₂TGGGT ACATGGAGGATGG(NNS)₂TAGATCTGTTGTACACAAAGTGGAGTAG-3′ (forward primer; SEQ ID NO: 4) and 5′-GAGCGTCCCAAAACCTTCTCAAG-3′ (reverse primer; SEQ ID NO: 5). The amplified piece of DNA was digested by AatII+MluI and inserted into pMJ7 backbone, which was digested with the same restriction enzymes.

### Example 3

### Selection for Zymogen Precursors in Yeast

Vip2 belongs to the family of actin ADP-ribosylating toxins. This NAD-dependent enzyme modifies monomeric actin at Arg 177 to block polymerization, leading to loss of cytoskeleton and cell death (Han et al., 1999). Actin is one of the most conserved proteins throughout the various species including mammalian, yeast and higher plants (Goodson and Hawse, 2002). Therefore, it was determined by the inventors that expression of a Vip2 ADP-ribosyltransferase in a model yeast organism, Saccharomyces cerevisae, was lethal to yeast cells.

Yeast cells could thus be transformed with a library of mutagenized/engineered Vip2 zymogen precursors (Vip2 variants) and yeast survivors comprising a defective Vip2 could be selected for. There are several benefits associated with using yeast for genetic selection. In the first place, yeast is likely to be the simplest, fast-growing organism whose viability depends on functional actin. Secondly, recombinant DNA technology and transformation systems in yeast are very well established. Finally, since actin ADP-ribosylation by Vip2 is most likely responsible for toxicity in transgenic corn, it is reasonable to assume that, as a eukaryote, yeast can mimic this situation to a certain extent and provide informative and predictive experimental data from engineering efforts in a much shorter time than afforded by transgenic plants.

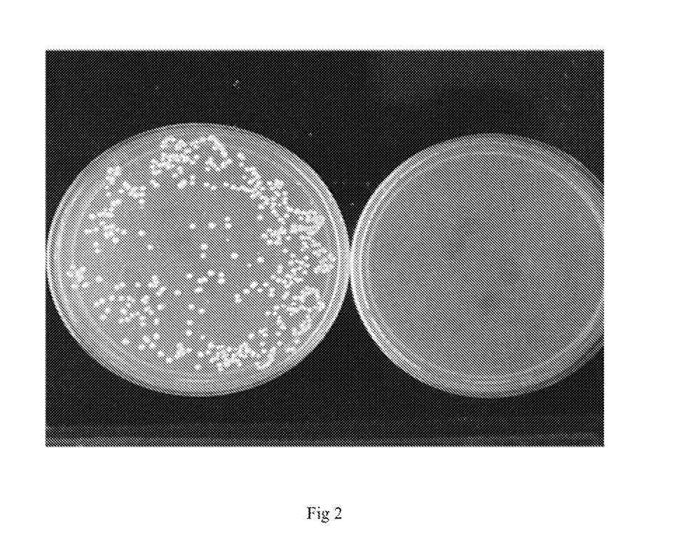

In order to test yeast cells for functional selection of Vip2 variants, both wild-type and the non-functional active-site mutant (E428G) genes were cloned into two yeast expression systems; high-copy number pYES2 and low-copy number p416GALS expression vectors. Both constructs were transformed into a laboratory strain of Saccharomyces cerevisae and selected under conditions supporting leaky expression from the Gal promoter (plates utilizing raffinose as a carbon source). While the E428G mutant gene in both expression systems produced many yeast transformants, there were no visible colonies after transformation of wild-type vip2 gene into yeast (FIG. 2). The E428G mutant vip2 gene thus served as a positive control to establish this genetic system as useful for functional selection of Vip2 variants. Thus, this simple genetic system can likely be adopted for rapid screening of functional significance of amino acid residues in any actin ADP-ribosyltransferase and for identification of critical residues. It was considered that an actin ADP-ribosyltransferase gene could be randomly mutagenized by any available in vitro or in vivo techniques and a pool of mutated genes gathered for transformation into yeast and selection of survivors. Sequencing of ADP-ribosyltransferase genes from yeast survivors should point out those amino acid residues that are crucial for enzyme function. This genetic system thus became a simple and powerful tool for selection of inactive enzyme variants and for implementing our propeptide strategy to repair Vip2 toxicity.

The propeptide library prepared in pMJ7 plasmid was transformed into Saccharomyces cerevisae INVSc1 using an EZ Yeast Transformation Kit (Zymo Research; Orange, Calif.) essentially following the manufacturer's instructions. Yeast survivors were selected under condition of leaky expression on SD-ura plates supplemented with 4% raffinose. The presence of raffinose as a carbon source in media does not induce or repress transcription from GAL promoter. Yeast minimal SD media and -ura dropout supplement were purchased from Clontech (Palo Alto, Calif.).

After yeast transformation, several colonies were selected under condition of “leaky” expression from a Gal promoter on plates supplemented with raffinose. Since the pMJ7 plasmid carrying the vip2 gene does not produce transformants on raffinose plates, any surviving colonies are expected to harbour zymogen precursors comprising an inactivated Vip2 toxin. In order to confirm the protective role of selected propeptide chains in Vip2 silencing, propeptides were recloned into pMJ7 plasmid backbone and retested in yeast transformation. Peptide from construct 4-4-12, VGWVPSRGEVFSLWVHGGWAR (SEQ ID NO: 6), was able to attenuate Vip2 activity to the extent that it allowed yeast colonies to emerge after transformation (although colonies exhibited signs of severe pathology, such as very slow growth). Furthermore, transformation efficiency with construct 4-4-12 was very low. Other peptides selected in the primary experiment did not pass the recloning test and appeared to be false positives. That is, colonies which originally survived after selection were most likely due to a novel mutation, deletion or rearrangement within vip2 gene itself, rather than direct protection by the C-terminally attached peptides.

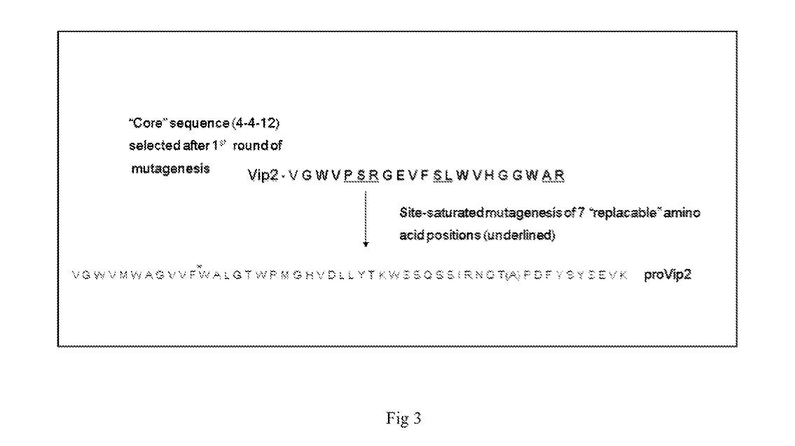

The spectrum of amino acids in the selected 4-4-12 propeptide (FIG. 3; SEQ ID NO: 6) does not correspond to the probability with which individual amino acids would be expected to appear in a random event. For example, in NN(G/C) randomization, the position of interest is changed to a complete set of 20 amino acids. Due to the disparity between residues like Met and Trp, which have a single codon, and residues like Leu, Arg, and Ser which have three codons, the probability with which individual amino acids appear in a completely unbiased library is different (e.g. Leu, Arg and Ser three times more frequently than Trp and Met). The presence of three tryptophans (Trp; W) in propeptides of surviving clones indicates their putative importance for propeptide function. Conversely, some multiple codon residues (Arg, Leu, Ser, Ala, Pro) have been selected with lower frequency, which may reflect their lower information content (higher replaceability, lower importance) in the selected peptide. These analyses allowed for identification of critical residues of the propeptide before attempting to improve its Vip2 silencing function by further mutagenesis. Thus, in one embodiment, the present invention encompasses a core sequence within the propeptide chain comprising the sequence X-X-W-X-X-X-X-X-X-X-X-X-X-W-X-X-X-X-W-X-X(SEQ ID NO: 7), where X is any amino acid.

The next set of mutagenesis experiments further decreased ADP-ribosylation activity of Vip2 by evolving the propeptide region of the selected proenzyme. The 4-4-12 clone propeptide coding sequence was used as a template for the next round of mutagenesis, in which blocks of several, presumably less important amino acids (PSR, SL, AR) were randomized simultaneously. As the parental, 4-4-12 proenzyme variant is able to form small colonies in yeast, a colony-size visual screen to identify propeptides with improved function was used to identify improved variants.

After transformation of Saccharomyces cerevisae with the mutagenized library, two healthy colonies were selected from the population of transformants on plates containing raffinose. Surprisingly, DNA sequencing of propeptide coding regions from both healthy survivors revealed the presence of 1) a single nucleotide transversion (A to T) responsible for Glu to Val substitution of the ninth amino acid in the propeptide region; and 2) a frameshift due to one nucleotide insertion after the eleventh amino acid (Phe) of the propeptide region thus extending the length of selected propeptides from the intended 21 amino acids to 49 amino acids. Part of these propeptides has thus been “acquired” from translated DNA sequence located downstream of the vip2 gene itself. Two selected propeptides have almost identical sequences, with only one conservative amino acid substitution (Thr vs. Ala; FIG. 3; SEQ ID NO: 8) at position number 39 of the polypeptide extension. Vip2 protein with the selected propeptide attached to the C-terminal end was designated proVip2-39T (wherein amino acid 449 of SEQ ID NO: 12 is Thr) and proVip2-39A (wherein amino acid 449 of SEQ ID NO: 12 is Ala). Removal of engineered propeptide-coding sequences from a proVip2 restored lethality of Vip2-ADP-ribosyltransferase in yeast, confirming an indispensable function of these sequences for silencing the enzymatic activity of Vip2 in yeast. Functionality of a propeptide sequence to compromise Vip2 toxicity was further confirmed by subcloning of propeptide sequences from low-copy number Vip2 plasmid backbone (pMJ7) into a high-copy number Vip2 plasmid backbone (pMJ5) and the ability of yeast to tolerate an even higher concentration of Vip2 in cells. These in vivo experiments clearly demonstrated that information necessary for yeast survival after transformation with Vip2 constructs resides on a propeptide sequence.

The in vivo selection in yeast demonstrated that the lethal effect of Vip2-ADP-ribosyltransferase in its zymogenic forms (proVip2) was compromised by C-terminally attached propeptide extensions. To validate this further, experiments were carried out to demonstrate that a Vip2 zymogen actually has a lower actin ADP-ribosylating activity than the wild-type Vip2 protein.

### Example 4

### Expression of vip2 Variants and Preparation of Protein Extracts

Proteins were expressed in E. coli BL21-Gold (DE3) cells. 100 ml of LB media supplemented with kanamycin (50 ug/ml) were inoculated with 1 ml of overnight culture and grown for 3 hours (OD600=0.5-0.8) at 37° C. before induction with 1 mM IPTG and grown for another 3.5 hours. Cells were collected by centrifugation and resuspended in 2 ml of 50 mM Tris-HCl, pH7.2, 50 mM NaCl. The cell suspension was lysed by use of the French press (Thermo Electron Corporation, Waltham, Mass.) and soluble proteins were recovered following centrifugation at 13.000×g for 15 minutes at 4° C.

### Example 5

### ADP-Ribosylation Assay

An in vitro ADP-ribosylation assay was carried out at 37° C. in a medium containing 10 mM Tris-HCl, pH 7.5, 1 mM CaCl₂, 0.5 mM ATP, 0.25 uM [³²P] NAD, 1 ug non-muscle actin (Cytoskeleton, Inc., Denver, Colo.) and 2.5 ng of enzyme in a total volume of 25 ul. The enzymatic reaction was stopped by adding SDS-PAGE sample buffer and boiling for 3 min. One half of the reaction volatile was subjected to SDS-PAGE, blotted onto 0.2 um PVDF membrane (Invitrogen, Carlsbad, Calif.) and processed by autoradiography.

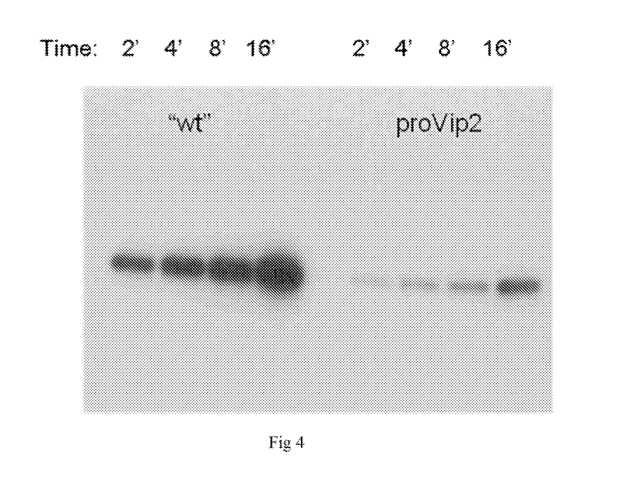

Vip2 and the engineered proVip2 proteins were expressed in Escherichia coli BL21(DE3) cells from the pET29a system, and the ADP-ribosylation reaction performed in vitro with a non-muscle actin. Kinetic ADP-ribosylation experiments with wild-type Vip2 and the proVip2 proteins, confirmed that the zymogenic proVip2 ADP-ribosylates actin to a lesser extent than the wild type protein (FIG. 4). Based on signal intensity, it was estimated from several independent kinetic experiments, that proVip2 exhibits less than 10% of actin ADP-ribosylation activity of its parental, “wt” form. Both engineered proVip2 proteins, proVip2-39T and proVip2-39A, ADP-ribosylate actin with the same efficiency. These in vitro experiments confirmed that the interpretation of the genetic selection strategy in yeast in terms of decreased ADP-ribosylation activity of Vip2 variants was correct.

Critically, even though proVip2 possesses less than 10% enzymatic activity of its native form, it retains potent toxicity to western corn rootworm larvae. Incorporation of the mixture of Vip1 helper protein and proVip2 culture extracts into artificial diet caused 100% mortality of corn rootworm larvae in 72 hours.

### Example 6

### Digestive Fate of Proteins in WCRW Larvae

A zymogen designed by the methods disclosed herein should have conditional activity whereby the zymogen is benign in a non-target organism or cell but toxic in target organism or cell. A particular, non-limiting example is provided by the “zymogenized” (polypeptide chain extended and malfunctional) Vip2 variants. First, the ADP-ribosylating activity of “zymogenized” Vip2 must be low enough to be tolerated by a plant host without symptoms of an aberrant phenotype. Survival of corn plants expressing the proVip2 zymogen precursors supports the first criterion. Second, the Vip2 zymogen should either possess enough residual enzymatic activity to be toxic to a plant pest such as corn rootworm, or have the potential to be convened into an enzymatically active form by a corn rootworm activator such as digestive proteases.

Therefore, a rootworm feeding assay was designed in which rootworm larvae were fed either Vip2 or its engineered zymogenic form, proVip2, in an artificial diet according essentially to the method of Marrone et al., (1985) and assess this aspect of its zymogen behavior. Because rootworm larvae possess a broad assortment of digestive enzymes (Bown et al., 2004), experiments were conducted to determine whether engineered proVip2 could be processed and possibly activated to the wild-type form in the rootworm digestive system.

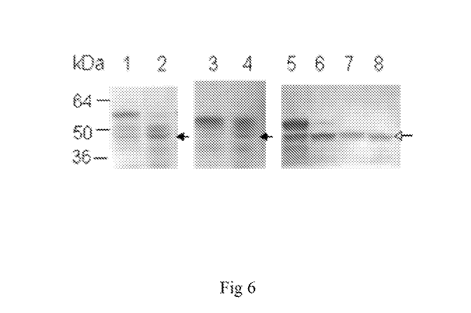

To facilitate visualization of protein after digestion, high doses of Vip2 proteins were incorporated into insect diet, achieved by using concentrated extracts from 10 ml of Escherichia coli BL21(DE3) cell culture. For Vip2 protein detection in whole body homogenates, rootworm larvae were fed on artificial diet comprising Vip2 protein or its zymogen for 30 or 90 minutes. After feeding, larvae were transferred into 1.5 ml Eppendorf tubes and stored at −80° C. until further processing. Larvae were homogenized in SDS-PAGE sample buffer containing 2× Complete Protease inhibitor cocktail (Roche Diagnostics) and heated to 100° C. for 5 minutes. After centrifugation, extracts from homogenized rootworm larvae were separated by SDS-PAGE and blotted onto PVDF membrane. Vip2 proteins were detected with rabbit anti-Vip2 antibody and visualized by HRP-labeled protein A using SuperSignal West Dura chemiluminiscent substrate (Pierce, Rockford, Ill.) or by donkey anti-rabbit antibody (Jackson ImmunoResearch Laboratories, West Grove, Pa.) followed by NBT/BCIP detection (Pierce). The resulting Western blot is shown in FIG. 6. Engineered Vip2 proenzymes, with or without an S-tag at the N-terminus (proVip2 and S-tag-proVip2), can be processed to a stable form of approximately the same size as wild-type Vip2 by western corn rootworm larvae. In the case of the N-terminally tagged Vip2 protein (S-tag-Vip2) processing involved removal of the S-tag as determined by lack of detection of the processed proteins with an S-protein antibody. These data support the interpretation that western corn rootworm larvae can activate the proVip2 molecule upon ingestion. Thus, the proVip2 zymogen is benign in a non-target organism or cell, for example a plant, but activated to a toxic protein in a target organism such as an insect pest.

For Vip2 protein detection in rootworm frass, rootworm larvae were fed artificial diet incorporated with Vip2 proteins for three days before excrement material was collected into 200 ul of enzyme assay buffer containing 10 mM Tris-HCl, pH7.5, 1 mM CaCl₂, 0.5 mM ATP. Collected soluble frass material was analyzed for the presence of enzymatic activity using the ADP-ribosylation assay described above and also examined by Western blot to assess proteolytic processing. Vip2 antigen was detected with rabbit anti-Vip2 antibody and visualized by Alkaline Phosphatase-conjugated donkey anti-rabbit antibody (Jackson ImmunoResearch Laboratories) followed by NBT/BCIP detection (Pierce).

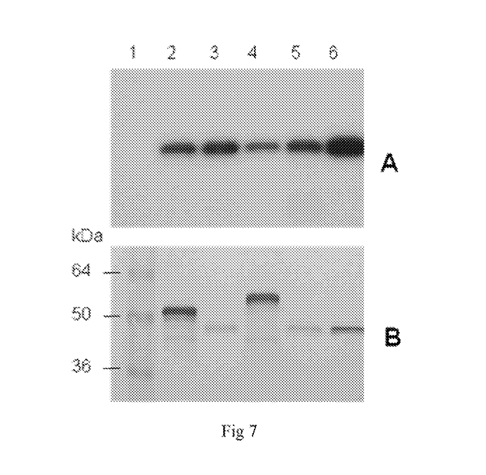

Since the receptor binding protein component of the binary toxin (Vip1) was not incorporated into the diet, feeding with Vip2 protein alone for a longer period of time (3 days) did not cause feeding inhibition or larval mortality. Analysis of proVip2 processing and enzymatic activity in frass from corn rootworm larvae again clearly demonstrated that enzyme precursors could be proteolytically processed to a stable, activated form of the protein. A substantially smaller amount of processed proVip2 protein recovered from rootworm frass had greater enzymatic activity than a much larger amount of undigested, control proVip2 protein (FIG. 7). These data therefore suggest that complete or partial removal of the engineered C-terminal peptide present in proVip2 by WCRW proteolytic activity has effectively “unmasked” the enzymatic activity needed to confer toxicity.

### Example 7

### Plant Transformation

Maize transformation was performed using the method essentially described by Negrotto at al., (2000). Two vectors for plant transformation were constructed, pNOV4500 (SEQ ID NO: 13) and pNOV4501 (SEQ ID NO: 14). The vectors contain the phosphomannose isomerase (PMI) gene for selection of transgenic maize lines (Negrotto et al., 2000). The expression cassettes comprises, in addition to the proVip2 gene, the MTL promoter (de Framond, 1994), extra-cytoplasmic (apoplast) targeting peptide from maize pathogenic related protein (Casacuberta et al., 1991) or maize chitinase secretion signal and 35S transcription terminator (Pietrzak et al., 1986).

ProVip2 transgenic corn did not show any symptoms of plant pathology under greenhouse conditions and was phenotypically unrecognizable from the control, untransformed plants.

In order to confirm the presence of proVip2 in transgenic corn an enzymatic ADP-ribosyltransferase assay with plant root extracts was performed. 250 mg of corn root material was homogenized in 200 μl of 50 mM sodium carbonate buffer, pH8.0 supplemented with 10 mM EDTA, 0.05% Tween 20, 0.05% Triton X-100, 100 mM NaCl, 1 mM AEBSF, 1 mM leupeptin and 1× Complete protease inhibitor cocktail (Roche Diagnostics, Indianapolis, Ind.). After homogenization, soluble protein extract was recovered by centrifugation at 12,000×g for 15 minutes. Ten microliters of root extract was used for the ADP-ribosylation assay.

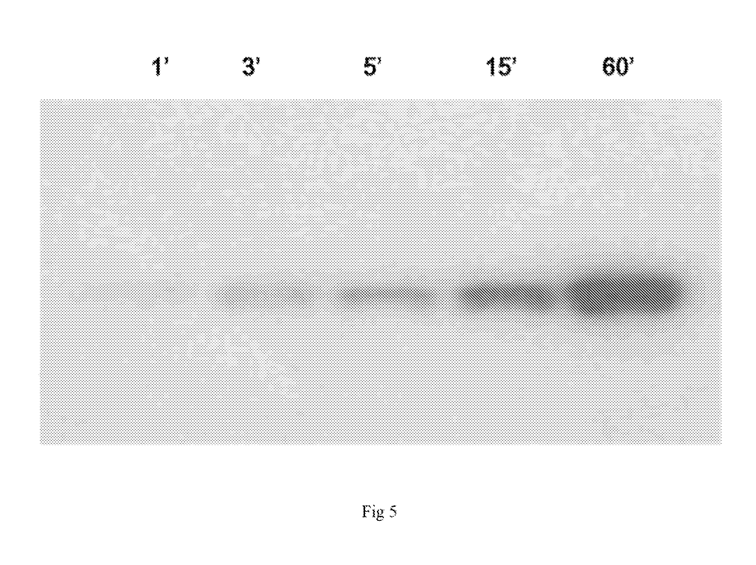

This sensitive labeling assay was able to detect ADP-ribosylation activity in root extracts from corn plants transformed with proVip2 (FIG. 5). Presence of the Vip2 antigen was also detected by an anti-Vip2 antibody confirming the ADP-ribosylating activity came from Vip2 protein.

### REFERENCES

Aktories, K., Barmann, M., Ohishi, I., Tsuyama, S., Jakobs, K. H. and Habermann, E. (1986) Nature, 322, 390-392
Aktories, K. and Wagner, A. (1992) Molecular Microbiology 6, 2905-2908
Branson, T. F. and Ortman, E. E. (1970) J. Econ. Entomol., 63, 800-803
Bown, D. P., Wilkinson, H. S., Jongsma, M. A. and Gatehouse, J. A. (2004) Insect Biochem. Molec. Biol. 34, 305-320
Casacuberta, J. M., Puigdomenech, P. and San Segundo, B. (1991) Plant Mol. Biol. 16 (4), 527-536
De Framond (1994) U.S. Pat. No. 5,466,785
Goodson, H. V. and Hawse, W. F. (2002) J. Cell. Sci. 115, 2619-1622
Han, S., Craig, J. A., Putnam, C. D., Carozzi, N. B., and Tainer, J. A. (1999) Nat. Struct. Biol., 6, 932-936
Jucovic, M, Walters, F. S., Warren, G. W., Palekar, N. V., Chen, J. S. (2008) Protein Engineering Design and Selection, 21(10):631-638
Lazure, C. (2002) Curr. Pharm. Des. 8, 511-531
Lee, Y. C., Miyata, Y., Terada, I., Ohta, T. and Matsuzawa, H. (1992) FEMS Microbiol. Lett. 92, 73-77
Marrone, P. G., Ferri, F. D., Mosley, T. R. and Meinke, L. J. (1985) J. Econ. Entom. 78(1): 290-293
Matsuoka, M. and Minami, E. (1989) Eur. J Biochem., 181(3):593-598
McQueney, M. S., Amegadzie, B. Y., D'Alessio, K., Hanning, C. R., McLaughlin, M. M., McNulty, D., Carr, S. A., Ijames, C., Kurdyla, J. and Jones, C. S. (1997) J. Biol. Chem. 272(21):13955-13960
Pietrzak, M., Shillito, R. D., Hohn, T. and Potrykus, I. (1986) Nucleic Acids Res. 14(14):5857-5868
Plainkum, P., Fuchs, S. M., Wiyakrutta, S. and Raines, R. T. (2003) Nature Struct. Biol. 10, 115-119
Rappuoli, R., Pizza, M. (1991) In Alouf, J. E. and Freer, J. H. (eds.), Sourcebook of Bacterial Protein Toxins. Academic Press, San Diego, Calif., pp. 1-21.
Stiles, B. G. and Wilkins, T. D. (1986) Infect. Immunol. 54, 683-688
Warren, G. W. et al. (1996) Novel pesticidal proteins and strains. World Intellectual Property Organization. Patent WO 96/10083
Warren, G. W. (1997) In Carozzi, N. B. and Koziel, M. G. (eds.), Advances in Insect Control: the role of transgenic plants. Gunpowder Square, London, UK, PP. 109-121
Warren, G. W., Koziel, M. G., Mullins, M. A., Nye, G. H., Carr, B., Desai, N. M. and Kostichka, K. (2000) U.S. Pat. No. 6,066,783
Warren, G. W., Koziel, M. G., Mullins, M. A., Nye, G. J., Carr, B. C., Desai, N. M., Kostichka, K., Duck, N. B. and Estruch, J. J. (2004) EP138261

1. An engineered zymogen of a toxic protein having a polypeptide chain extension fused to a C-terminus or a N-terminus of the toxic protein, wherein the zymogen is benign in a non-target organism or cell and wherein the zymogen is converted to a toxic protein when the zymogen is in a target organism or cell.

2. The zymogen of claim 1, wherein the toxic protein is an ADP-ribosyltransferase.

3. The zymogen of claim 2, wherein the ADP-ribosyltransferase ribosylates actin.

4. The zymogen of claim 2, wherein the ADP-ribosyltransferase comprises an amino acid sequence with at least 69% sequence identity to SEQ ID NO:9 and wherein the ADP-ribosyltransferase has a catalytic residue that corresponds to E428 of SEQ ID NO:9 and NAD binding residues that correspond to Y307, R349, E355, F397, and R400 of SEQ ID NO:9.

5. The zymogen of claim 2, wherein the ADP-ribosyltransferase is insecticidal.

6. The zymogen of claim 2, wherein the ADP-ribosyltransferase is a Vip2 toxin.

7. The zymogen of claim 6, wherein the Vip2 toxin is selected from a group consisting of SEQ ID NO:9, 10, 15, 16, 17, 18, and 19.

8. The zymogen of claim 2, wherein the polypeptide extension comprises an amino acid sequence of at least 21 residues long and having a tryptophan (Trp; W) residue at position 3, 14, and 19.

9. The zymogen of claim 8, wherein the polypeptide extension comprises SEQ ID NO:6.

10. The zymogen of claim 2, wherein the polypeptide extension comprises SEQ ID NO:8.

11. The zymogen of claim 8, 9, or 10, wherein the polypeptide chain extension is fused to the C-terminus of the ADP-ribosyltransferase.

12. The zymogen of claim 1, wherein the non-target organism or cell is a plant, a plant cell, or a yeast cell.

13. The zymogen of claim 12, wherein the plant or plant cell is selected from the group consisting of sorghum, wheat, tomato, cole crops, cotton, rice, soybean, sugar beet, sugarcane, tobacco, barley, oilseed rape, and maize.

14. The zymogen of claim 13, wherein the plant or plant cell is maize.

15. The zymogen of claim 12, wherein the yeast cell is Saccharomyces cerevisae.

16. The zymogen of claim 1, wherein the zymogen comprises SEQ ID NO:11 or SEQ ID NO:12.

17. An isolated nucleic acid molecule comprising a nucleotide sequence encoding a zymogen according to claim 1.

18. A recombinant vector comprising the isolated nucleic acid molecule of claim 17.

19. A transgenic plant or plant cell comprising the nucleic acid molecule of claim 17.

20. The transgenic plant of claim 19 that is a maize plant or maize plant cell.

21. A yeast cell comprising the isolated nucleic acid molecule of claim 17.

22. The yeast cell of claim 21, wherein the yeast is Saccharomyces cerevisae.

23. A method of making a zymogen of a toxic protein, the method comprising the steps of:
(a) designing a polypeptide chain which extends from a terminus of the toxic protein;
(b) making a library of expression plasmids which will express a precursor including the polypeptide chain upon transformation into a genetic system;
expressing the precursors in a genetic system that is naturally susceptible to the toxic protein;
(c) recovering organisms or cells of a genetic system which survive step (c);
isolating the precursors from the organisms or cells of step (d);
testing the precursors for biological activity against a target organism or cell; and
identifying the biologically active precursors as zymogens.

24. The method according to claim 23, wherein the toxic protein is an ADP-ribosyltransferase.

25. The method according to claim 24, wherein the ADP-ribosyltransferase ribosylates actin.

26. The method according to claim 24, wherein the ADP-ribosyltransferase is insecticidal.

27. The method according to claim 24, wherein the ADP-ribosyltransferase is a Vip2 toxin.

28. The method according to claim 27, wherein the Vip2 toxin is selected from a group consisting of SEQ ID NO:9, 10, 15, 16, 17, 18, and 19.

29. The method according to claim 23, wherein the library comprises random amino acid sequences of at least 21 residues and having a tryptophan (Trp; W) residue at position 3, 14, and 19.

30. The method according to claim 23, wherein the genetic system is a eukaryotic organism or cell.

31. The method according to claim 30, wherein the genetic system is yeast.

32. The method according to claim 31, wherein yeast is Saccharomyces cerevisae.

33. The method according to claim 23, wherein the target organism or cell is eukaryotic or prokaryotic.

34. The method according to claim 33, wherein the target organism or cell is an insect or insect cell.

35. The method according to claim 34, wherein the insect or insect cell is in the genus Diabrotica.

36. The method according to claim 35, wherein the insect organism or cell is Diabrotica virgifera (western corn rootworm), D. longicornis (northern rootworm), or D. virgifera zeae (Mexican corn rootworm).

37. The method according to claim 23, wherein the zymogen is biologically active in the target cell.

38. A genetic system that allows for efficient identification of an engineered zymogen of a toxic protein, wherein the zymogen is benign in a non-target organism or cell and wherein the zymogen is converted to a toxic protein when the zymogen is in a target organism or cell.

39. The genetic system of claim 38, wherein the engineered zymogen comprises a polypeptide chain extending from the C-terminus or N-terminus of the toxic protein.

40. The genetic system of claim 38 that is yeast.

41. The genetic system of claim 40, wherein the yeast is Saccharomyces cerevisae.

42. The genetic system of claim 38, wherein the target organism or cell is a pathogenic cell or organism.

43. The genetic system of claim 38, wherein the toxic protein is an ADP-ribosyltransferase.

44. The genetic system of claim 43, wherein the ADP-ribosyltransferase ribosylates actin.


SEQUENCE LISTING (23 sequences)

SEQ ID NO: 1 - DNA, 31 bp, Artificial Sequence
  Chemically synthesized
tatacatatgctgcagaacctgaagatcacc

SEQ ID NO: 2 - DNA, 36 bp, Artificial Sequence
  Chemically synthesized
tctagatgcatgctcgagctaggacgtcagcagggt

SEQ ID NO: 3 - DNA, 36 bp, Artificial Sequence
  Chemically synthesized
tctagatgcatgctcgagtcacttcacttcactgta

SEQ ID NO: 4 - DNA, 104 bp, Artificial Sequence
  Chemically synthesized
gatcaggacgtccgtaggatgggtannsnnsnnsggtgaagtattcnnsnnstgggtacatggaggatggnnsnnstagatctgttgtacacaaagtggagtag

SEQ ID NO: 5 - DNA, 23 bp, Artificial Sequence
  Chemically synthesized
gagcgtcccaaaaccttctcaag

SEQ ID NO: 6 - protein, 21 aa, Artificial sequence
  Chemically synthesized
VGWVPSRGEVFSLWVHGGWAR

SEQ ID NO: 7 - protein, 21 aa, Artificial Sequece
  Chemically synthesized
XXWXXXXXXXXXXWXXXXWXX

SEQ ID NO: 8 - protein, 50 aa, Artificial Sequence
  Chemically synthesized
VGWVMWAGVVFWALGTWRMGHVDLLYTKWSSQSSIRNQXAPDFYSYSEVK

SEQ ID NO: 9 - protein, 462 aa, Bacillus cereus
MKRMEGKLFMVSKKLQVVTKTVLLSTVFSISLLNNEVIKAEQLNINSQSKYTNLQNLKITDKVEDFKEDKEKAKEWGKEKEKEWKLTATEKGKMNNFLDNKNDIKTNYKEITFSMAGSFEDEIKDLKEIDKMFDKTNLSNSIITYKNVEPTTIGFNKSLTEGNTINSDAMAQFKEQFLDRDIKFDSYLDTHLTAQQVSSKERVILKVTVPSGKGSTTPTKAGVILNNSEYKMLIDNGYMVHVDKVSKVVKKGVECLQIEGTLKKSLDFKNDINAEAHSWGMKNYEEWAKDLTDSQREALDGYARQDYKEINNYLRNQGGSGNEKLDAQIKNISDALGKKPIPENITVYRWCGMPEFGYQISDPLPSLKDFEEQFLNTIKEDKGYMSTSLSSERLAAFGSRKIILRLQVPKGSTGAYLSAIGGFASEKEILLDKDSKYHIDKVTEVIIKGVKRYVVDATLLTN

SEQ ID NO: 10 - protein, 410 aa, Artificial Sequence
  Truncated Vip2 without bacterial secretion signal.
MLQNLKITDKVEDFKEDKEKAKEWGKEKEKEWKLTATEKGKMNNFLDNKNDIKTNYKEITFSMAGSFEDEIKDLKEIDKMFDKTNLSNSIITYKNVEPTTIGFNKSLTEGNTINSDAMAQFKEQFLDRDIKFDSYLDTHLTAQQVSSKERVILKVTVPSGKGSTTPTKAGVILNNSEYKMLIDNGYMVHVDKVSKVVKKGVECLQIEGTLKKSLDFKNDINAEAHSWGMKNYEEWAKDLTDSQREALDGYARQDYKEINNYLRNQGGSGNEKLDAQIKNISDALGKKPIPENITVYRWCGMPEFGYQISDPLPSLKDFEEQFLNTIKEDKGYMSTSLSSERLAAFGSRKIILRLQVPKGSTGAYLSAIGGFASEKEILLDKDSKYHIDKVTEVIIKGVKRYVVDATLLTX

SEQ ID NO: 11 - protein, 431 aa, Artificial Sequence
  Chemiocally synthesized
MLQNLKITDKVEDFKEDKEKAKEWGKEKEKEWKLTATEKGKMNNFLDNKNDIKTNYKEITFSMAGSFEDEIKDLKEIDKMFDKTNLSNSIITYKNVEPTTIGFNKSLTEGNTINSDAMAQFKEQFLDRDIKFDSYLDTHLTAQQVSSKERVILKVTVPSGKGSTTPTKAGVILNNSEYKMLIDNGYMVHVDKVSKVVKKGVECLQIEGTLKKSLDFKNDINAEAHSWGMKNYEEWAKDLTDSQREALDGYARQDYKEINNYLRNQGGSGNEKLDAQIKNISDALGKKPIPENITVYRWCGMPEFGYQISDPLPSLKDFEEQFLNTIKEDKGYMSTSLSSERLAAFGSRKIILRLQVPKGSTGAYLSAIGGFASEKEILLDKDSKYHIDKVTEVIIKGVKRYVVDATLLTXVGWVPSRGEVFSLWVHGGWAR

SEQ ID NO: 12 - protein, 460 aa, Artificial Sequence
  Chemically synthesized
MLQNLKITDKVEDFKEDKEKAKEWGKEKEKEWKLTATEKGKMNNFLDNKNDIKTNYKEITFSMAGSFEDEIKDLKEIDKMFDKTNLSNSIITYKNVEPTTIGFNKSLTEGNTINSDAMAQFKEQFLDRDIKFDSYLDTHLTAQQVSSKERVILKVTVPSGKGSTTPTKAGVILNNSEYKMLIDNGYMVHVDKVSKVVKKGVECLQIEGTLKKSLDFKNDINAEAHSWGMKNYEEWAKDLTDSQREALDGYARQDYKEINNYLRNQGGSGNEKLDAQIKNISDALGKKPIPENITVYRWCGMPEFGYQISDPLPSLKDFEEQFLNTIKEDKGYMSTSLSSERLAAFGSRKIILRLQVPKGSTGAYLSAIGGFASEKEILLDKDSKYHIDKVTEVIIKGVKRYVVDATLLTXVGWVMWAGVVFWALGTWRMGHVDLLYTKWSSQSSIRNQXAPDFYSYSEVK

SEQ ID NO: 13 - DNA, 13299 bp, Artificial Sequence
  Chemically synthesized
cgcgccagcttgcacatgacaacaattgtaagaggatggagaccacaacgatccaacaatacttctgcgacgggctgtgaagtatagagaagttaaacgcccaaaagccattgtgtttggaatttttagttattctatttttcatgatgtatcttcctctaacatgccttaatttgcaaatttggtataactactgattgaaaatatatgtatgtaaaaaaatactaagcatatttgtgaagctaaacatgatgttatttaagaaaatatgttgttaacagaataagattaatatcgaaatggaaacatctgtaaattagaatcatcttacaagctaagagatgttcacgctttgagaaacttcttcagatcatgaccgtagaagtagctctccaagactcaacgaaggctgctgcaattccacaaatgcatgacatgcatccttgtaaccgtcgtcgccgctataaacacggataactcaattccctgctccatcaatttagaaatgagcaagcaagcacccgatcgctcaccccatatgcaccaatctgactcccaagctctgtttcgcattagtaccgccagcactccacctatagctaccaattgagacctttccagcctaagcagatcgattgatcgttagagtcaaagagttggtggtacgggtactttaactaccatggaatgatggggcgtgatgtagagcggaaagcgcctccctacgcggaacaacaccctcgccatgccgctcgactacagcctcctcctcgtcggcgccacaacgagggagcccgtggtcgcagccaccgaccagcatgtctctgtgtcctcgtccgacctcgacatgtcatggcaaacagtcggacgccagcaccagactgacgacatgagtctctgaagagcccgccacctagaaagatccgagccctgctgctggtagtggtaaccattttcgtcgcgctgacgcggagagcgagaggccagaaatttatagcgactgacgctgtggcaggcacgctatcggaggttacgacgtggcgggtcactcgacgcggagttcacaggtcctatccttgcatcgctcggcgcggagtttacggggacttatccttacgacgtgctctaaggttgcgataacgggcggaggaaggcgtgtggcgtgcggagacggtttatacacgtagtgtgcgggagtgtgtttcgtagacgcgggaaagcacgacgacttacgaaggttagtggaggaggaggacacactaaaatcaggacgcaagaaactcttctattatagtagtagagaagagattataggagtgtgggttgattctaaagaaaatcgacgcaggacaaccgtcaaaacgggtgctttaatatagtagatatatatatatagagagagagagaaagtacaaaggatgcatttgtgtctgcatatgatcggagtattactaacggccgtcgtaagaaggtccatcatgcgtggagcgagcccatttggttggttgtcaggccgcagttaaggcctccatatatgattgtcgtcgggcccataacagcatctcctccaccagtttattgtaagaataaattaagtagagatatttgtcgtcgggcagaagaaacttggacaagaagaagaagcaagctaggccaatttcttgccggcaagaggaagatagtggcctctagtttatatatcggcgtgatgatgatgctcctagctagaaatgagagaagaaaaacggacgcgtgtttggtgtgtgtcaatggcgtccatccttccatcagatcagaacgatgaaaaagtcaagcacggcatgcatagtatatgtatagcttgttttagtgtggctttgctgagacgaatgaaagcaacggcgggcatatttttcagtggctgtagctttcaggctgaaagagacgtggcatgcaataattcagggaattcgtcagccaattgaggtagctagtcaacttgtacattggtgcgagcaattttccgcactcaggagggctagtttgagagtccaaaaactataggagattaaagaggctaaaatcctctccttatttaattttaaataagtagtgtatttgtattttaactcctccaacccttccgattttatggctctcaaactagcattcagtctaatgcatgcatgcttggctagaggtcgtatggggttgttaatagcatagctagctacaagttaaccgggtcttttatatttaataaggacaggcaaagtattacttacaaataaagaataaagctaggacgaactgctggattattactaaatcgaaatggacgtaatattccaggcaagaataattgttcgatcaggagacaagtggggcattggaccggttcttgcaagcaagagcctatggcgtggtgacacggcgcgttgcccatacatcatgcctccatcgatgatccatcctcacttgctataaaaagaggtgtccatggtgctcaagctcagccaagcaaataagacgacttgtttcattgattcttcaagagatcgagcttcttttgcaccacaaggtcgaggatccaccatgatgagagccctggcgtggtggccatgctggcccgccttcttcgctgtgcccgctcgcgccctgcagaacctgaagatcaccgacaaggtggaggacttcaaggaggacaaggagaaggccaaggagtggggcaaggagaaggagaaggagtggaagcttaccgccaccgagaagggcaagatgaacaacttcctggacaacaagaacgacatcaagaccaactacaaggagatcaccttcagcatggccggcagcttcgaggacgagatcaaggacctgaaggagatcgacaagatgttcgacaagaccaacctgagcaacagcatcatcacctacaagaacgtggagcccaccaccatcggcttcaacaagagcctgaccgagggcaacaccatcaacagcgacgccatggcccagttcaaggagcagttcctggaccgcgacatcaagttcgacagctacctggacacccacctgaccgcccagcaggtgagcagcaaggagcgcgtgatcctgaaggtgaccgtccccagcggcaagggcagcaccacccccaccaaggccggcgtgatcctgaacaacagcgagtacaagatgctgatcgacaacggctacatggtgcacgtggacaaggtgagcaaggtggtgaagaagggcgtggagtgcctccagatcgagggcaccctgaagaagagtctagacttcaagaacgacatcaacgccgaggcccacagctggggcatgaagaactacgaggagtgggccaaggacctgaccgacagccagcgcgaggccctggacggctacgcccgccaggactacaaggagatcaacaactacctgcgcaaccagggcggcagcggcaacgagaagctggacgcccagatcaagaacatcagcgacgccctcggcaagaagcccatccccgagaacatcaccgtgtaccgatggtgcggcatgcccgagttcggctaccagatcagcgaccccctgcccagcctgaaggacttcgaggagcagttcctgaacaccatcaaggaggacaagggctacatgagcaccagcctgagcagcgagcgcctggccgccttcggcagccgcaagatcatcctgcgcctgcaggtgcccaagggcagcactggtgcctacctgagcgccatcggcggcttcgccagcgagaaggagatcctgctggataaggacagcaagtaccacatcgacaaggtgaccgaggtgatcatcaagggcgtgaagcgctacgtggtggacgccaccctgctgacgtccgtaggatgggtaatgtgggcgggtgtagtattctgggcgctgggtacatggaggatggggcacgtagatctgttgtacacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccaggggtacccggggatcctctagagtcgaccatggtgatcactgcaggcatgcaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctgctagccctgcaggaaatttaccggtgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccccgggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctaaatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaattaattcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattggg

SEQ ID NO: 14 - DNA, 13317 bp, Artificial Sequence
  Chemically synthesized
cgcgccagcttgcacatgacaacaattgtaagaggatggagaccacaacgatccaacaatacttctgcgacgggctgtgaagtatagagaagttaaacgcccaaaagccattgtgtttggaatttttagttattctatttttcatgatgtatcttcctctaacatgccttaatttgcaaatttggtataactactgattgaaaatatatgtatgtaaaaaaatactaagcatatttgtgaagctaaacatgatgttatttaagaaaatatgttgttaacagaataagattaatatcgaaatggaaacatctgtaaattagaatcatcttacaagctaagagatgttcacgctttgagaaacttcttcagatcatgaccgtagaagtagctctccaagactcaacgaaggctgctgcaattccacaaatgcatgacatgcatccttgtaaccgtcgtcgccgctataaacacggataactcaattccctgctccatcaatttagaaatgagcaagcaagcacccgatcgctcaccccatatgcaccaatctgactcccaagctctgtttcgcattagtaccgccagcactccacctatagctaccaattgagacctttccagcctaagcagatcgattgatcgttagagtcaaagagttggtggtacgggtactttaactaccatggaatgatggggcgtgatgtagagcggaaagcgcctccctacgcggaacaacaccctcgccatgccgctcgactacagcctcctcctcgtcggcgccacaacgagggagcccgtggtcgcagccaccgaccagcatgtctctgtgtcctcgtccgacctcgacatgtcatggcaaacagtcggacgccagcaccagactgacgacatgagtctctgaagagcccgccacctagaaagatccgagccctgctgctggtagtggtaaccattttcgtcgcgctgacgcggagagcgagaggccagaaatttatagcgactgacgctgtggcaggcacgctatcggaggttacgacgtggcgggtcactcgacgcggagttcacaggtcctatccttgcatcgctcggcgcggagtttacggggacttatccttacgacgtgctctaaggttgcgataacgggcggaggaaggcgtgtggcgtgcggagacggtttatacacgtagtgtgcgggagtgtgtttcgtagacgcgggaaagcacgacgacttacgaaggttagtggaggaggaggacacactaaaatcaggacgcaagaaactcttctattatagtagtagagaagagattataggagtgtgggttgattctaaagaaaatcgacgcaggacaaccgtcaaaacgggtgctttaatatagtagatatatatatatagagagagagagaaagtacaaaggatgcatttgtgtctgcatatgatcggagtattactaacggccgtcgtaagaaggtccatcatgcgtggagcgagcccatttggttggttgtcaggccgcagttaaggcctccatatatgattgtcgtcgggcccataacagcatctcctccaccagtttattgtaagaataaattaagtagagatatttgtcgtcgggcagaagaaacttggacaagaagaagaagcaagctaggccaatttcttgccggcaagaggaagatagtggcctctagtttatatatcggcgtgatgatgatgctcctagctagaaatgagagaagaaaaacggacgcgtgtttggtgtgtgtcaatggcgtccatccttccatcagatcagaacgatgaaaaagtcaagcacggcatgcatagtatatgtatagcttgttttagtgtggctttgctgagacgaatgaaagcaacggcgggcatatttttcagtggctgtagctttcaggctgaaagagacgtggcatgcaataattcagggaattcgtcagccaattgaggtagctagtcaacttgtacattggtgcgagcaattttccgcactcaggagggctagtttgagagtccaaaaactataggagattaaagaggctaaaatcctctccttatttaattttaaataagtagtgtatttgtattttaactcctccaacccttccgattttatggctctcaaactagcattcagtctaatgcatgcatgcttggctagaggtcgtatggggttgttaatagcatagctagctacaagttaaccgggtcttttatatttaataaggacaggcaaagtattacttacaaataaagaataaagctaggacgaactgctggattattactaaatcgaaatggacgtaatattccaggcaagaataattgttcgatcaggagacaagtggggcattggaccggttcttgcaagcaagagcctatggcgtggtgacacggcgcgttgcccatacatcatgcctccatcgatgatccatcctcacttgctataaaaagaggtgtccatggtgctcaagctcagccaagcaaataagacgacttgtttcattgattcttcaagagatcgagcttcttttgcaccacaaggtcgaggatccaccatggaggcatccaacaagctcgcagtcttgctcctgtggctggtcatggcagctgccactgccgtgcacccttcctactctctgcagaacctgaagatcaccgacaaggtggaggacttcaaggaggacaaggagaaggccaaggagtggggcaaggagaaggagaaggagtggaagcttaccgccaccgagaagggcaagatgaacaacttcctggacaacaagaacgacatcaagaccaactacaaggagatcaccttcagcatggccggcagcttcgaggacgagatcaaggacctgaaggagatcgacaagatgttcgacaagaccaacctgagcaacagcatcatcacctacaagaacgtggagcccaccaccatcggcttcaacaagagcctgaccgagggcaacaccatcaacagcgacgccatggcccagttcaaggagcagttcctggaccgcgacatcaagttcgacagctacctggacacccacctgaccgcccagcaggtgagcagcaaggagcgcgtgatcctgaaggtgaccgtccccagcggcaagggcagcaccacccccaccaaggccggcgtgatcctgaacaacagcgagtacaagatgctgatcgacaacggctacatggtgcacgtggacaaggtgagcaaggtggtgaagaagggcgtggagtgcctccagatcgagggcaccctgaagaagagtctagacttcaagaacgacatcaacgccgaggcccacagctggggcatgaagaactacgaggagtgggccaaggacctgaccgacagccagcgcgaggccctggacggctacgcccgccaggactacaaggagatcaacaactacctgcgcaaccagggcggcagcggcaacgagaagctggacgcccagatcaagaacatcagcgacgccctcggcaagaagcccatccccgagaacatcaccgtgtaccgatggtgcggcatgcccgagttcggctaccagatcagcgaccccctgcccagcctgaaggacttcgaggagcagttcctgaacaccatcaaggaggacaagggctacatgagcaccagcctgagcagcgagcgcctggccgccttcggcagccgcaagatcatcctgcgcctgcaggtgcccaagggcagcactggtgcctacctgagcgccatcggcggcttcgccagcgagaaggagatcctgctggataaggacagcaagtaccacatcgacaaggtgaccgaggtgatcatcaagggcgtgaagcgctacgtggtggacgccaccctgctgacgtccgtaggatgggtaatgtgggcgggtgtagtattctgggcgctgggtacatggaggatggggcacgtagatctgttgtacacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccaggggtacccggggatcctctagagtcgaccatggtgatcactgcaggcatgcaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctgctagccctgcaggaaatttaccggtgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccccgggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctaaatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaattaattcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattggg

SEQ ID NO: 15 - protein, 462 aa, Bacillus thuringiensis
MKRMEGKLFMVSTKLQAVTKAVLLSTVLSISLLNNEVIKAEQLNMNSQNKYTNFENLKITDKVEDFKEDKEKAKEWGKEKEKEWKLTATEKGKMNNFLDNKNDIKTNYKEITFSMAGSFEDEIKDLKEIDKIFDKANLSSPIITYKNVEPATIGFNKSLTEGNTINSDAMAQFKEQFLDRDIKFDSYLDTHLTVQQVSSKERVILKVKVPSGKGSTTPTKAGIILNNSEYKMLIDNGYMVHVDKVSKVVKKGVECLQVEGTLKKSLDFKNDINAGAHSWGMKNYEEWAKDLTDLQREALDGYARQDYKEINNYLRNQGGNGNEKLDAQIKNISDALGKKPIPENITVYRWCGMPEFGYQISDPLPSLKDFEEQFLNTIKEDKGYMSTSLSSERLAAFGSRKIILRLQVPKGSTGAYLSAIGGFANEKEILLDKDSKYHIDKVTEVIIKGVKRYVVDATLLTN

SEQ ID NO: 16 - protein, 462 aa, Bacillus thuringiensis
MQRMEGKLFMVSKKLQAVTKTVLLSTVLSISLLNNEEVKAEQLNINSQNKYTNFQNLKITDNAEDFKEDKEKAKEWGEEKEKEWKLTATEKGKMNNFLDNKNDIKTNYKEITFSMAGSFEDEIKDLKEIDKIFDKANLSSSIITYKNVEPATIGFNKSLTEGNTINSDAMAQFKEQFLGKDMKFDSYLDTHLTAHQVSSKKRVILKVTVPSGKGSTTPTKAGVILTNNEYKMLIDNGYVLHVDKVSKVVKKGMECLQVEGTLKKSLDFKNDINAEAHSWGMKIYEDWAKNLTASQREALDGYARQDYKEINNYLRNQGGSGNEKLDAQIKNISDALGKKPIPENITVYRWCGMPEFGYQISDPLPSLKDFEEQFLNTIKEDKGYMSTSLSSERLAAFGSRKIILRLQVPKGSTGAYLSAIGGFASEKEILLDKDSKYHIDKATEVIIKGVKRYVVDATLLTN

SEQ ID NO: 17 - protein, 462 aa, Bacillus thuringiensis
MKRMEGKLFMVSRKLQLVTKALLFSTVLSIPLLNNEEVKAEHLNLNSQSKYPSFQNQKITDNAEDFKEDKEKAKEWGEVKEKEWKLTATEKRKINDFLNDTNKIKTNYKEITFSMAGSFEDELKDLKEIDKMFDKANLSSSIITYKNVEPATIGFNKSLTEGNTINSDVMAQFKEQFLGKDIKFDSYLDTHLTVQQVSSKERVILKVTVPSGKGSTNPTKAGVILDGNEPKMLIDNGYVLHVDKVSKVVKKGLECLQVEGTLKKSLDFKNDISAKAHSWGMKNYEEWAANLTDSQRKALDGYARQDYKKINDYLRNQGGSGNEQLDAQIKNISETLNNKPIPENITVYRWCGMPEFGYQISEPLPALKDFEWEFLNTIKEDKGYISTSLSSERLAAFGSRKIILRLQIPKGSKGAYLSAIGGFANEKEILLDKDSKYHINKITEVVIKGIKRYVVDATLLTN

SEQ ID NO: 18 - protein, 457 aa, Bacillus thuringiensis
MIVIIFTNVKGGNELKKNFYKNLICMSALLLAMPISSNVTYAYGSEKVDYLVKTTNNTEDFKEDKEKAKEWGKEKEKEWKLTVTEKTRMNNFLDNKNDIKKNYKEITFSMAGSFEDEIKDLKEIDKMFDKANLSSSIVTYKNVEPSTIGFNKPLTEGNTINTDVQAQFKEQFLGKDIKFDSYLDTHLTAQNVSSKERIILQVTVPSGKGSTIPTKAGVILNNNEYKMLIDNGYVLHVDNISKVVKKGYECLQIQGTLKKSLDFKNDINAEAHRWGMKNYEGWAKNLTDPQREALDGYARQDYKQINDYLRNQGGSGNEKLDTQIKNISEALEKQPIPENITVYRWCGMAEFGYQISDPLPSLKEMEEKFLNTMKEDKGYMSTSLSSERLSAFGSRKFILRLQVPKGSTGAYLSAIGGFASEKEILIDKDSNYHIDKITEVVIKGVKRYVVDATLLTK

SEQ ID NO: 19 - protein, 460 aa, Bacillus thuringiensis
MKRMEERLFMVSKKLQLITKTLVFSTVLSIPLLNNSEIKAEQLNMNSQIKYPNFQNINIADKPVDFKEDKEKAREWGKEKEKEWKLTATEKGKINDFLDDKDGLKTKYKEINFSKNFEYETELKELEKINTMLDKANLTNSIVTYKNVEPTTIGFNQSLIEGNQINAEAQQKFKEQFLGQDIKFDSYLDMHLTEQNVSSKERVILKVTVPSGKGSTPTKAGVVLNNNEYKMLIDNGYVLHVENITKVVKKGQECLQVEGTLKKSLDFKNDSDGKGDSWGKKNYKEWSDTLTTDQRKDLNDYGARGYTEINKYLREGGTGNTELEEKIKNISDALEKNPIPENITVYRYCGMAEFGYPIKPEAPSVQDFEERFLDTIKEEKGYMSTSLSSDATSFGARKIILRLQVPKGSSGAYVAGLDGFKPAEKEILIDKGSKYRIDKVTEVVVKGTRKLVVDATLLTK

SEQ ID NO: 20 - protein, 463 aa, Clostrium difficile
MKKFRKHKRISNCISILLILYLTLGGLLPNNIYAQDLQSYSEKVCNTTYKAPIERPEDFLKDKEKAKEWERKEAERIEQKLERSEKEALESYKKDSVEISKYSQTRNYFYDYQIEANSREKEYKELRNAISKNKIDKPMYVYYFESPEKFAFNKVIRTENQNEISLEKFNEFKETIQNKLFKQDGFKDISLYEPGKGDEKPTPLLMHLKLPRNTGMLPYTNTNNVSTLIEQGYSIKIDKIVRIVIDGKHYIKAEASVVSSLDFKDDVSKGDSWGKANYNDWSNKLTPNELADVNDYMRGGYTAINNYLISNGPVNNPNPELDSKITNIENALKREPIPTNLTVYRRSGPQEFGLTLTSPEYDFNKLENIDAFKSKWEGQALSYPNFISTSIGSVNMSAFAKRKIVLRITIPKGSPGAYLSAIPGYAGEYEVLLNHGSKFKINKIDSYKDGTITKLIVDATLIP

SEQ ID NO: 21 - protein, 454 aa, Clostridium perfringens
MKKVNKSISVFLILYLILTSSFPSYTYAQDLQIASNYITDRAFIERPEDFLKDKENAIQWEKKEAERVEKNLDTLEKEALELYKKDSEQISNYSQTRQYFYDYQIESNPREKEYKNLRNAISKNKIDKPINVYYFESPEKFAFNKEIRTENQNEISLEKFNELKETIQDKLFKQDGFKDVSLYEPGNGDEKPTPLLIHLKLPKNTGMLPYINSNDVKTLIEQDYSIKIDKIVRIVIEGKQYIKAEASIVNSLDFKDDVSKGDLWGKENYSDWSNKLTPNELADVNDYMRGGYTAINNYLISNGPLNNPNPELDSKVNNIENALKLTPIPSNLIVYRRSGPQEFGLTLTSPEYDFNKIENIDAFKEKWEGKVITYPNFISTSIGSVNMSAFAKRKIILRINIPKDSPGAYLSAIPGYAGEYEVLLNHGSKFKINKVDSYKDGTVTKLILDATLIN

SEQ ID NO: 22 - protein, 459 aa, Clostridium spiroforme
MKKYKNNCISILLMLFLILTGLFPNTVFAQGAQSYDFRTINNIANYSAIERPEDFLKDKEKAKDWERKEAERIEKNLEKSEREALESYKKDAVEISKYSQVRNYFYDYPIEANTREKEYKELKNAVSKNKIDKPMYVYYFESPEKFAFNKEIRAESQNEISLERFNEFKATIQDKLFKQDGFKDISLYEPGNGDKKSTPLLIHLKLPKDTGMLPYSNSNDVSTLIEQGYSIKIDKIVRIVLEGKQYIKAEASVVSCLDFKDDVSKGDSWGKANYSDWSNKLSSDELAGVNDYMRGRYTAINNYLIANGPTNNPNAELDAKINNIENALKREPIPANLVVYRRSGPQEFGLTLSSPEYDFNKVENIDAFKEKWEGQTLSYPNFVSTSIGSVNMSAFAKRKIVLRISIPKNSPGAYLSAIPGYAGEYEVLLNHGSKFKISKIDSYKDGTTTKLIVDRTLID

SEQ ID NO: 23 - protein, 431 aa, Clostridium botulinum
MPIIKEPIDFINKPESEAQKWGKEEEKRWFTKLNNLEEVAVNQLKTKEDKTKIDNFSTDILFSSLTAIEIMKEDENQNLFDVERIREALLKNTLDREVIGYVNFTPKELGINFSIRDVELNRDISDEILDKVRQQIINQEYTKFSFVSLGLNDNSIDESIPVIVKTRVPTTFNYGVLNNKETVSLLLNQGFSIIPESAIITTIKGKDYILIEGSLSQELDFYNKGSEAWGEKNYGDYVSKLSQEQLGALEGYLHSDYKAINSYLRNNRVPNNDELNKKIELISSALSVKPIPETLIAYRRVDGIPFDLPSDFSFDKKENGEIIADKTKLNEFIDKWTGKEIENLSFSSTSLKSTPLSFSKSRFIFRLRLSEGTIGAFIYGFSGFQDEQEILLNKNSTFKIFRITPITSIINRVTKMTQVVIDAEVIQNKEI